SEQUENCE LISTING (109 sequences)

SEQ ID NO: 1 - protein, 4 aa, Homo Sapiens
HHQK

SEQ ID NO: 2 - protein, 7 aa, Homo Sapiens
CGHHQKG

SEQ ID NO: 3 - protein, 120 aa, Mus Musculus
QVQLVQSGAEVKKPGASVKMSCKASGYSFTSYWINWVKQRPGQGLEWIGDVHPGRGVSTYNAKFKSRATLTLDTSISTAYMELSSLRSEDTAVYYCSRSHGNTYWFFDVWGQGTTVTVSS

SEQ ID NO: 4 - protein, 112 aa, Mus Musculus
DVLMTQSPLSLPVTLGQPASISCRSSQSIVHSNGNTYLEWYQQRPGQSPRLLIYKVSNRFSGVPDRFSGSGSGTDFTLKISRVEAEDVGVYYCFQGSHVPFTFGQGTKLEIK

SEQ ID NO: 5 - protein, 8 aa, Mus Musculus
GYSFTSYW

SEQ ID NO: 6 - protein, 9 aa, Mus Musculus
VHPGRGVST

SEQ ID NO: 7 - protein, 13 aa, Mus Musculus
SRSHGNTYWFFDV

SEQ ID NO: 8 - protein, 11 aa, Mus Musculus
QSIVHSNGNTY

SEQ ID NO: 9 - protein, 3 aa, Mus Musculus
KVS

SEQ ID NO: 10 - protein, 9 aa, Mus Musculus
FQGSHVPFT

SEQ ID NO: 11 - protein, 120 aa, Artificial Sequence
  Synthetic Construct
QVQLVQSGAEVKKPGASVKMSCKASGYSFTSYWINWVKQRPGQGLEWIGDVHPGRGVSTYNAKFKSRATLTLDTSISTAYMELSSLRSEDTAVYYCARGALSPYWFFDVWGQGTTVTVSS

SEQ ID NO: 12 - protein, 120 aa, Artificial Sequence
  Synthetic Construct
QVQLVQSGAEVKKPGASVKMSCKASGYSFTSYWINWVKQRPGQGLEWIGDVHPGRGVSTYNAKFKSRATLTLDTSISTAYMELSSLRSEDTAVYYCARSKTPVYWFFDVWGQGTTVTVSS

SEQ ID NO: 13 - protein, 120 aa, Artificial Sequence
  Synthetic Construct
QVQLVQSGAEVKKPGASVKMSCKASGYSFTSYWINWVKQRPGQGLEWIGDVHPGRGVSTYNAKFKSRATLTLDTSISTAYMELSSLRSEDTAVYYCARVSPTGYWFFDVWGQGTTVTVSS

SEQ ID NO: 14 - protein, 120 aa, Artificial Sequence; identical to SEQ ID NO: 11
  Synthetic Construct

SEQ ID NO: 15 - protein, 120 aa, Artificial Sequence
  Synthetic Construct
QVQLVQSGAEVKKPGASVKMSCKASGYSFTSYWINWVKQRPGQGLEWIGDVHPGRGVSTYNAKFKSRATLTLDTSISTAYMELSSLRSEDTAVYYCSRSSPFNYWFFDVWGQGTTVTVSS

SEQ ID NO: 16 - protein, 120 aa, Artificial Sequence
  Synthetic Construct
QVQLVQSGAEVKKPGASVKMSCKASGYSFTSYWINWVKQRPGQGLEWIGDVHPGRGVSTYNAKFKSRATLTLDTSISTAYMELSSLRSEDTAVYYCARSPSSTYWFFDVWGQGTTVTVSS

SEQ ID NO: 17 - protein, 120 aa, Artificial Sequence
  Synthetic Construct
QVQLVQSGAEVKKPGASVKMSCKASGYSFTSYWINWVKQRPGQGLEWIGDVHPGRGVSTYNAKFKSRATLTLDTSISTAYMELSSLRSEDTAVYYCARGAMSPYWFFDVWGQGTTVTVSS

SEQ ID NO: 18 - protein, 120 aa, Artificial Sequence
  Synthetic Construct
QVQLVQSGAEVKKPGASVKMSCKASGYSFTSYWINWVKQRPGQGLEWIGDVHPGRGVSTYNAKFKSRATLTLDTSISTAYMELSSLRSEDTAVYYCAKSPQQSYWFFDVWGQGTTVTVSS

SEQ ID NO: 19 - protein, 120 aa, Artificial Sequence
  Synthetic Construct
QVQLVQSGAEVKKPGASVKMSCKASGYSFTSYWINWVKQRPGQGLEWIGDVHPGRGVSTYNAKFKSRATLTLDTSISTAYMELSSLRSEDTAVYYCARSTTTGYWFFDVWGQGTTVTVSS

SEQ ID NO: 20 - protein, 120 aa, Artificial Sequence
  Synthetic Construct
QVQLVQSGAEVKKPGASVKMSCKASGYSFTSYWINWVKQRPGQGLEWIGDVHPGRGVSTYNAKFKSRATLTLDTSISTAYMELSSLRSEDTAVYYCARSSAQDYWFFDVWGQGTTVTVSS

SEQ ID NO: 21 - protein, 120 aa, Artificial Sequence
  Synthetic Construct
QVQLVQSGAEVKKPGASVKMSCKASGYSFTSYWINWVKQRPGQGLEWIGDVHPGRGVSTYNAKFKSRATLTLDTSISTAYMELSSLRSEDTAVYYCARSVGDTYWFYDVWGQGTTVTVSS

SEQ ID NO: 22 - protein, 120 aa, Artificial Sequence
  Synthetic Construct
QVQLVQSGAEVKKPGASVKMSCKASGYSFTSYWINWVKQRPGQGLEWIGDVHPGRGVSTYNAKFKSRATLTLDTSISTAYMELSSLRSEDTAVYYCAKSDASGYWFFDVWGQGTTVTVSS

SEQ ID NO: 23 - protein, 120 aa, Artificial Sequence
  Synthetic Construct
QVQLVQSGAEVKKPGASVKMSCKASGYSFTSYWINWVKQRPGQGLEWIGDVHPGRGVSTYNAKFKSRATLTLDTSISTAYMELSSLRSEDTAVYYCSRSHGNTPWFLDDWGQGTTVTVSS

SEQ ID NO: 24 - protein, 120 aa, Artificial Sequence
  Synthetic Construct
QVQLVQSGAEVKKPGASVKMSCKASGYSFTSYWINWVKQRPGQGLEWIGDVHPGRGVSTYNAKFKSRATLTLDTSISTAYMELSSLRSEDTAVYYCARSSGGRYWFFDVWGQGTTVTVSS

SEQ ID NO: 25 - protein, 120 aa, Artificial Sequence
  Synthetic Construct
QVQLVQSGAEVKKPGASVKMSCKASGYSFTSYWINWVKQRPGQGLEWIGDVHPGRGVSTYNAKFKSRATLTLDTSISTAYMELSSLRSEDTAVYYCSRSHGNSPAFFDVWGQGTTVTVSS

SEQ ID NO: 26 - protein, 120 aa, Artificial Sequence
  Synthetic Construct
QVQLVQSGAEVKKPGASVKMSCKASGYSFTSYWINWVKQRPGQGLEWIGDVHPGRGVSTYNAKFKSRATLTLDTSISTAYMELSSLRSEDTAVYYCARSMSQNYWFFDVWGQGTTVTVSS

SEQ ID NO: 27 - protein, 120 aa, Artificial Sequence
  Synthetic Construct
QVQLVQSGAEVKKPGASVKMSCKASGYSFTSYWINWVKQRPGQGLEWIGDVHPGRGVSTYNAKFKSRATLTLDTSISTAYMELSSLRSEDTAVYYCARSSVHGYWFFDVWGQGTTVTVSS

SEQ ID NO: 28 - protein, 120 aa, Artificial Sequence
  Synthetic Construct
QVQLVQSGAEVKKPGASVKMSCKASGYSFTSYWINWVKQRPGQGLEWIGDVHPGRGVSTYNAKFKSRATLTLDTSTSTAYMELSSLRSEDTAVYYCSRSHGNRPWFYDFWGQGTTVTVSS

SEQ ID NO: 29 - protein, 120 aa, Artificial Sequence
  Synthetic Construct
QVQLVQSGAEVKKPGASVKMSCKASGYSFTSYWINWVKQRPGQGLEWIGDVHPGRGVSTYNAKFKSRATLTLDTSISTAYMELSSLRSEDTAVYYCARSSHKGYWFFDVWGQGTTVTVSS

SEQ ID NO: 30 - protein, 120 aa, Artificial Sequence
  Synthetic Construct
QVQLVQSGAEVKKPGASVKMSCKASGYSFTSYWINWVKQRPGQGLEWIGDVHPGRGVSTYNAKFKSRATLTLDTSISTAYMELSSLRSEDTAVYYCARVSPMSYWFFDVWGQGTTVTVSS

SEQ ID NO: 31 - protein, 13 aa, Artificial Sequence
  Synthetic Construct
ARGALSPYWFFDV

SEQ ID NO: 32 - protein, 13 aa, Artificial Sequence
  Synthetic Construct
ARSKTPVYWFFDV

SEQ ID NO: 33 - protein, 13 aa, Artificial Sequence
  Synthetic Construct
ARVSPTGYWFFDV

SEQ ID NO: 34 - protein, 13 aa, Artificial Sequence
  Synthetic Construct
ARGALSPYWFFDV

SEQ ID NO: 35 - protein, 13 aa, Artificial Sequence
  Synthetic Construct
SRSSPFNYWFFDV

SEQ ID NO: 36 - protein, 13 aa, Artificial Sequence
  Synthetic Construct
ARSPSSTYWFFDV

SEQ ID NO: 37 - protein, 13 aa, Artificial Sequence
  Synthetic Construct
ARGAMSPYWFFDV

SEQ ID NO: 38 - protein, 13 aa, Artificial Sequence
  Synthetic Construct
AKSPQQSYWFFDV

SEQ ID NO: 39 - protein, 13 aa, Artificial Sequence
  Synthetic Construct
ARSTTTGYWFFDV

SEQ ID NO: 40 - protein, 13 aa, Artificial Sequence
  Synthetic Construct
ARSSAQDYWFFDV

SEQ ID NO: 41 - protein, 13 aa, Artificial Sequence
  Synthetic Construct
ARSVGDTYWFYDV

SEQ ID NO: 42 - protein, 13 aa, Artificial Sequence
  Synthetic Construct
AKSDASGYWFFDV

SEQ ID NO: 43 - protein, 13 aa, Artificial Sequence
  Synthetic Construct
SRSHGNTPWFLDD

SEQ ID NO: 44 - protein, 13 aa, Artificial Sequence
  Synthetic Construct
ARSSGGRYWFFDV

SEQ ID NO: 45 - protein, 13 aa, Artificial Sequence
  Synthetic Construct
SRSHGNSPAFFDV

SEQ ID NO: 46 - protein, 13 aa, Artificial Sequence
  Synthetic Construct
ARSMSQNYWFFDV

SEQ ID NO: 47 - protein, 13 aa, Artificial Sequence
  Synthetic Construct
ARSSVHGYWFFDV

SEQ ID NO: 48 - protein, 13 aa, Artificial Sequence
  Synthetic Construct
SRSHGNRPWFYDF

SEQ ID NO: 49 - protein, 13 aa, Artificial Sequence
  Synthetic Construct
ARSSHKGYWFFDV

SEQ ID NO: 50 - protein, 13 aa, Artificial Sequence
  Synthetic Construct
ARVSPMSYWFFDV

SEQ ID NO: 51 - DNA, 360 bp, Artificial Sequence
  Synthetic Construct
caggtccaactggtgcagtctggggctgaggtgaagaagcctggggcttcagtgaagatgtcctgcaaggcttctggctacagcttcaccagctactggataaactgggtgaagcagaggcctggacaaggccttgagtggattggagatgtgcatcctggtagaggcgtgtccacatacaatgctaagttcaagagcagagccacactgactctggacacatccataagcacagcctacatggagctcagcagcctgagatctgaggacacggcggtctattactgtgccaggggcgccctcagtccctactggttttttgacgtctggggccaaggcaccacggtcaccgtctcctca

SEQ ID NO: 52 - DNA, 360 bp, Artificial Sequence
  Synthetic Construct
caggtccaactggtgcagtctggggctgaggtgaagaagcctggggcttcagtgaagatgtcctgcaaggcttctggctacagcttcaccagctactggataaactgggtgaagcagaggcctggacaaggccttgagtggattggagatgtgcatcctggtagaggcgtgtccacatacaatgctaagttcaagagcagagccacactgactctggacacatccataagcacagcctacatggagctcagcagcctgagatctgaggacacggcggtctattactgtgccaggtccaagacgccggtctactggttttttgacgtctggggccaaggcaccacggtcaccgtctcctca

SEQ ID NO: 53 - DNA, 360 bp, Artificial Sequence
  Synthetic Construct
caggtccaactggtgcagtctggggctgaggtgaagaagcctggggcttcagtgaagatgtcctgcaaggcttctggctacagcttcaccagctactggataaactgggtgaagcagaggcctggacaaggccttgagtggattggagatgtgcatcctggtagaggcgtgtccacatacaatgctaagttcaagagcagagccacactgactctggacacatccataagcacagcctacatggagctcagcagcctgagatctgaggacacggcggtctattactgtgccagggtctcgccgacgggctactggttttttgacgtctggggccaaggcaccacggtcaccgtctcctca

SEQ ID NO: 54 - DNA, 360 bp, Artificial Sequence
  Synthetic Construct
caggtccaactggtgcagtctggggctgaggtgaagaagcctggggcttcagtgaagatgtcctgcaaggcttctggctacagcttcaccagctactggataaactgggtgaagcagaggcctggacaaggccttgagtggattggagatgtgcatcctggtagaggcgtgtccacatacaatgctaagttcaagagcagagccacactgactctggacacatccataagcacagcctacatggagctcagcagcctgagatctgaggacacggcggtctattactgtgccaggggcgcgttgtctccctactggttttttgacgtctggggccaaggcaccacggtcaccgtctcctca

SEQ ID NO: 55 - DNA, 360 bp, Artificial Sequence
  Synthetic Construct
caggtccaactggtgcagtctggggctgaggtgaagaagcctggggcttcagtgaagatgtcctgcaaggcttctggctacagcttcaccagctactggataaactgggtgaagcagaggcctggacaaggccttgagtggattggagatgtgcatcctggtagaggcgtgtccacatacaatgctaagttcaagagcagagccacactgactctggacacatccataagcacagcctacatggagctcagcagcctgagatctgaggacacggcggtctattactgttccaggtccagcccgtttaactactggttttttgacgtctggggccaaggcaccacggtcaccgtctcctca

SEQ ID NO: 56 - DNA, 360 bp, Artificial Sequence
  Synthetic Construct
caggtccaactggtgcagtctggggctgaggtgaagaagcctggggcttcagtgaagatgtcctgcaaggcttctggctacagcttcaccagctactggataaactgggtgaagcagaggcctggacaaggccttgagtggattggagatgtgcatcctggtagaggcgtgtccacatacaatgctaagttcaagagcagagccacactgactctggacacatccataagcacagcctacatggagctcagcagcctgagatctgaggacacggcggtctattactgtgccaggtccccgtcctcgacctactggttttttgacgtctggggccaaggcaccacggtcaccgtctcctca

SEQ ID NO: 57 - DNA, 360 bp, Artificial Sequence
  Synthetic Construct
caggtccaactggtgcagtctggggctgaggtgaagaagcctggggcttcagtgaagatgtcctgcaaggcttctggctacagcttcaccagctactggataaactgggtgaagcagaggcctggacaaggccttgagtggattggagatgtgcatcctggtagaggcgtgtccacatacaatgctaagttcaagagcagagccacactgactctggacacatccataagcacagcctacatggagctcagcagcctgagatctgaggacacggcggtctattactgtgccaggggcgccatgtctccctactggttttttgacgtctggggccaaggcaccacggtcaccgtctcctca

SEQ ID NO: 58 - DNA, 360 bp, Artificial Sequence
  Synthetic Construct
caggtccaactggtgcagtctggggctgaggtgaagaagcctggggcttcagtgaagatgtcctgcaaggcttctggctacagcttcaccagctactggataaactgggtgaagcagaggcctggacaaggccttgagtggattggagatgtgcatcctggtagaggcgtgtccacatacaatgctaagttcaagagcagagccacactgactctggacacatccataagcacagcctacatggagctcagcagcctgagatctgaggacacggcggtctattactgtgccaagtccccccagcagtcctactggttttttgacgtctggggccaaggcaccacggtcaccgtctcctca

SEQ ID NO: 59 - DNA, 360 bp, Artificial Sequence
  Synthetic Construct
caggtccaactggtgcagtctggggctgaggtgaagaagcctggggcttcagtgaagatgtcctgcaaggcttctggctacagcttcaccagctactggataaactgggtgaagcagaggcctggacaaggccttgagtggattggagatgtgcatcctggtagaggcgtgtccacatacaatgctaagttcaagagcagagccacactgactctggacacatccataagcacagcctacatggagctcagcagcctgagatctgaggacacggcggtctattactgtgccaggtccacgacgacgggctactggttttttgacgtctggggccaaggcaccacggtcaccgtctcctca

SEQ ID NO: 60 - DNA, 360 bp, Artificial Sequence
  Synthetic Construct
caggtccaactggtgcagtctggggctgaggtgaagaagcctggggcttcagtgaagatgtcctgcaaggcttctggctacagcttcaccagctactggataaactgggtgaagcagaggcctggacaaggccttgagtggattggagatgtgcatcctggtagaggcgtgtccacatacaatgctaagttcaagagcagagccacactgactctggacacatccataagcacagcctacatggagctcagcagcctgagatctgaggacacggcggtctattactgtgccaggtcctccgcgcaggactactggttttttgacgtctggggccaaggcaccacggtcaccgtctcctca

SEQ ID NO: 61 - DNA, 360 bp, Artificial Sequence
  Synthetic Construct
caggtccaactggtgcagtctggggctgaggtgaagaagcctggggcttcagtgaagatgtcctgcaaggcttctggctacagcttcaccagctactggataaactgggtgaagcagaggcctggacaaggccttgagtggattggagatgtgcatcctggtagaggcgtgtccacatacaatgctaagttcaagagcagagccacactgactctggacacatccataagcacagcctacatggagctcagcagcctgagatctgaggacacggcggtctattactgtgccaggtccgtgggggatacctactggttttatgacgtctggggccaaggcaccacggtcaccgtctcctca

SEQ ID NO: 62 - DNA, 360 bp, Artificial Sequence
  Synthetic Construct
caggtccaactggtgcagtctggggctgaggtgaagaagcctggggcttcagtgaagatgtcctgcaaggcttctggctacagcttcaccagctactggataaactgggtgaagcagaggcctggacaaggccttgagtggattggagatgtgcatcctggtagaggcgtgtccacatacaatgctaagttcaagagcagagccacactgactctggacacatccataagcacagcctacatggagctcagcagcctgagatctgaggacacggcggtctattactgtgccaagtccgacgcctcgggctactggttttttgacgtctggggccaaggcaccacggtcaccgtctcctca

SEQ ID NO: 63 - DNA, 360 bp, Artificial Sequence
  Synthetic Construct
caggtccaactggtgcagtctggggctgaggtgaagaagcctggggcttcagtgaagatgtcctgcaaggcttctggctacagcttcaccagctactggataaactgggtgaagcagaggcctggacaaggccttgagtggattggagatgtgcatcctggtagaggcgtgtccacatacaatgctaagttcaagagcagagccacactgactctggacacatccataagcacagcctacatggagctcagcagcctgagatctgaggacacggcggtctattactgtagcagatctcatggtaacaccccttggtttctcgacgactggggccaaggcaccacggtcaccgtctcctca

SEQ ID NO: 64 - DNA, 360 bp, Artificial Sequence
  Synthetic Construct
caggtccaactggtgcagtctggggctgaggtgaagaagcctggggcttcagtgaagatgtcctgcaaggcttctggctacagcttcaccagctactggataaactgggtgaagcagaggcctggacaaggccttgagtggattggagatgtgcatcctggtagaggcgtgtccacatacaatgctaagttcaagagcagagccacactgactctggacacatccataagcacagcctacatggagctcagcagcctgagatctgaggacacggcggtctattactgtgccaggtcctccgggggtcgctactggttttttgacgtctggggccaaggcaccacggtcaccgtctcctca

SEQ ID NO: 65 - DNA, 360 bp, Artificial Sequence
  Synthetic Construct
caggtccaactggtgcagtctggggctgaggtgaagaagcctggggcttcagtgaagatgtcctgcaaggcttctggctacagcttcaccagctactggataaactgggtgaagcagaggcctggacaaggccttgagtggattggagatgtgcatcctggtagaggcgtgtccacatacaatgctaagttcaagagcagagccacactgactctggacacatccataagcacagcctacatggagctcagcagcctgagatctgaggacacggcggtctattactgtagcagatctcatggtaactccccggcgtttttcgacgtctggggccaaggcaccacggtcaccgtctcctca

SEQ ID NO: 66 - DNA, 360 bp, Artificial Sequence
  Synthetic Construct
caggtccaactggtgcagtctggggctgaggtgaagaagcctggggcttcagtgaagatgtcctgcaaggcttctggctacagcttcaccagctactggataaactgggtgaagcagaggcctggacaaggccttgagtggattggagatgtgcatcctggtagaggcgtgtccacatacaatgctaagttcaagagcagagccacactgactctggacacatccataagcacagcctacatggagctcagcagcctgagatctgaggacacggcggtctattactgtgccaggtccatgtcccagaactactggttttttgacgtctggggccaaggcaccacggtcaccgtctcctca

SEQ ID NO: 67 - DNA, 360 bp, Artificial Sequence
  Synthetic Construct
caggtccaactggtgcagtctggggctgaggtgaagaagcctggggcttcagtgaagatgtcctgcaaggcttctggctacagcttcaccagctactggataaactgggtgaagcagaggcctggacaaggccttgagtggattggagatgtgcatcctggtagaggcgtgtccacatacaatgctaagttcaagagcagagccacactgactctggacacatccataagcacagcctacatggagctcagcagcctgagatctgaggacacggcggtctattactgtgccaggtcctcggtgcatggctactggttttttgacgtctggggccaaggcaccacggtcaccgtctcctca

SEQ ID NO: 68 - DNA, 360 bp, Artificial Sequence
  Synthetic Construct
caggtccaactggtgcagtctggggctgaggtgaagaagcctggggcttcagtgaagatgtcctgcaaggcttctggctacagcttcaccagctactggataaactgggtgaagcagaggcctggacaaggccttgagtggattggagatgtgcatcctggtagaggcgtgtccacatacaatgctaagttcaagagcagagccacactgactctggacacatccacaagcacagcctacatggagctcagcagcctgagatctgaggacacggcggtctattactgtagcagatctcatggtaaccgcccgtggttttacgacttctggggccaaggcaccacggtcaccgtctcctca

SEQ ID NO: 69 - DNA, 360 bp, Artificial Sequence
  Synthetic Construct
caggtccaactggtgcagtctggggctgaggtgaagaagcctggggcttcagtgaagatgtcctgcaaggcttctggctacagcttcaccagctactggataaactgggtgaagcagaggcctggacaaggccttgagtggattggagatgtgcatcctggtagaggcgtgtccacatacaatgctaagttcaagagcagagccacactgactctggacacatccataagcacagcctacatggagctcagcagcctgagatctgaggacacggcggtctattactgtgccaggtcctcccacaagggctactggttttttgacgtctggggccaaggcaccacggtcaccgtctcctca

SEQ ID NO: 70 - DNA, 360 bp, Artificial Sequence
  Synthetic Construct
caggtccaactggtgcagtctggggctgaggtgaagaagcctggggcttcagtgaagatgtcctgcaaggcttctggctacagcttcaccagctactggataaactgggtgaagcagaggcctggacaaggccttgagtggattggagatgtgcatcctggtagaggcgtgtccacatacaatgctaagttcaagagcagagccacactgactctggacacatccataagcacagcctacatggagctcagcagcctgagatctgaggacacggcggtctattactgtgccagggtctccccgatgagctactggttttttgacgtctggggccaaggcaccacggtcaccgtctcctca

SEQ ID NO: 71 - DNA, 360 bp, Mus Musculus
caggtccaactggtgcagtctggggctgaggtgaagaagcctggggcttcagtgaagatgtcctgcaaggcttctggctacagcttcaccagctactggataaactgggtgaagcagaggcctggacaaggccttgagtggattggagatgtgcatcctggtagaggcgtgtccacatacaatgctaagttcaagagcagagccacactgactctggacacatccataagcacagcctacatggagctcagcagcctgagatctgaggacacggcggtctattactgtagcagatcccatggtaacacctactggttttttgacgtctggggccaaggcaccacggtcaccgtctcctca

SEQ ID NO: 72 - DNA, 336 bp, Mus Musculus
gatgttctgatgacccaatctccactctccctgcctgtcacccttggacagccggcctccatctcttgcagatctagtcagagcattgtacatagtaatggaaacacctatttagaatggtaccagcagaggccaggccagtctccaaggctgctgatctacaaagtttccaaccgattttctggggtcccagacaggttcagtggcagtggatcagggacagatttcacactcaagatcagcagagtggaggctgaggatgttggagtttattactgctttcaaggttcacatgttcctttcacttttggccaagggaccaagctggagatcaaa

SEQ ID NO: 73 - DNA, 984 bp, Mus Musculus
gcttccaccaagggcccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaatgtagatcacaagcccagcaacaccaaggtggacaagagagttgagtccaaatatggtcccccatgcccaccatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaatga

SEQ ID NO: 74 - protein, 327 aa, Mus Musculus
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 75 - DNA, 324 bp, Mus Musculus
cgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 76 - protein, 107 aa, Mus Musculus [sequence omitted]

SEQ ID NO: 77 - protein, 42 aa, Homo Sapiens [sequence omitted]

SEQ ID NO: 78 - protein, 9 aa, Homo Sapiens
AEFRHDSGY

SEQ ID NO: 79 - protein, 14 aa, Homo Sapiens
VHHQKLVFFAEDVG

SEQ ID NO: 80 - protein, 6 aa, Homo Sapiens
HQKLVF

SEQ ID NO: 81 - protein, 10 aa, Homo Sapiens
HQKLVFFAED

SEQ ID NO: 82 - protein, 7 aa, Homo Sapiens
EVHHQKL

SEQ ID NO: 83 - protein, 14 aa, Homo Sapiens
VHHAEDVFFAEDVG

SEQ ID NO: 84 - protein, 5 aa, Mus Musculus
SYWIN

SEQ ID NO: 85 - protein, 17 aa, Mus Musculus
DVHPGRGVSTYNAKFKS

SEQ ID NO: 86 - protein, 11 aa, Mus Musculus
GALSPYWFFDV

SEQ ID NO: 87 - protein, 16 aa, Mus Musculus
RSSQSIVHSNGNTYLE

SEQ ID NO: 88 - protein, 7 aa, Mus Musculus
KVSNRFS

SEQ ID NO: 89 - protein, 9 aa, Mus Musculus
FQGSHVPFT

SEQ ID NO: 90 - protein, 11 aa, Artificial Sequence
  Synthetic Construct
GALSPYWFFDV

SEQ ID NO: 91 - protein, 11 aa, Artificial Sequence
  Synthetic Construct
SKTPVYWFFDV

SEQ ID NO: 92 - protein, 11 aa, Artificial Sequence
  Synthetic Construct
VSPTGYWFFDV

SEQ ID NO: 93 - protein, 11 aa, Artificial Sequence
  Synthetic Construct
GALSPYWFFDV

SEQ ID NO: 94 - protein, 11 aa, Artificial Sequence
  Synthetic Construct
SPSSTYWFFDV

SEQ ID NO: 95 - protein, 11 aa, Artificial Sequence
  Synthetic Construct
SPSSTYWFFDV

SEQ ID NO: 96 - protein, 11 aa, Artificial Sequence
  Synthetic Construct
GAMSPYWFFDV

SEQ ID NO: 97 - protein, 11 aa, Artificial Sequence
  Synthetic Construct
SPQQSYWFFDV

SEQ ID NO: 98 - protein, 11 aa, Artificial Sequence
  Synthetic Construct
STTTGYWFFDV

SEQ ID NO: 99 - protein, 11 aa, Artificial Sequence
  Synthetic Construct
SSAQDYWFFDV

SEQ ID NO: 100 - protein, 11 aa, Artificial Sequence
  Synthetic Construct
SVGDTYWFYDV

SEQ ID NO: 101 - protein, 11 aa, Artificial Sequence
  Synthetic Construct
SDASGYWFFDV

SEQ ID NO: 102 - protein, 11 aa, Artificial Sequence
  Synthetic Construct
SHGNTPWFLDD

SEQ ID NO: 103 - protein, 11 aa, Artificial Sequence
  Synthetic Construct
SSGGRYWFFDV

SEQ ID NO: 104 - protein, 11 aa, Artificial Sequence
  Synthetic Construct
SHGNSPAFFDV

SEQ ID NO: 105 - protein, 11 aa, Artificial Sequence
  Synthetic Construct
SMSQNYWFFDV

SEQ ID NO: 106 - protein, 11 aa, Artificial Sequence
  Synthetic Construct
SSVHGYWFFDV

SEQ ID NO: 107 - protein, 11 aa, Artificial Sequence
  Synthetic Construct
SHGNRPWFYDF

SEQ ID NO: 108 - protein, 11 aa, Artificial Sequence
  Synthetic Construct
SSHKGYWFFDV

SEQ ID NO: 109 - protein, 11 aa, Artificial Sequence
  Synthetic Construct
VSPMSYWFFDV

(19) United States
(12) Patent Application Publication
(10) Pub. No.: US 2023/0141177 A1
(43) Pub. Date: May 11, 2023

(54) ANTIBODIES TO MISFOLDED AMYLOID BETA

(71) Applicants: THE UNIVERSITY OF BRITISH COLUMBIA, Vancouver (CA); PROMIS NEUROSCIENCES INC., Toronto (CA)

(72) Inventors: Neil R. Cashman, Vancouver (CA); Johanne Kaplan, Sherborn, MA (US); Ebrima Gibbs, Port Moody (CA)

(21) Appl. No.: 17/916,302

(22) PCT Filed: Mar. 31, 2021
(86) PCT No.: PCT/CA2021/050431

§ 371 Date: Sep. 30, 2022

Related U.S. Application Data
(60) Provisional application No. 63/002,899, filed on Mar. 31, 2020.

(51) Int. Cl. C07K 16/18; A61K 47/68; A61P 25/28; G01N 33/68

(52) U.S. Cl. CPC C07K 16/18; A61K 47/6843; A61P 25/28; G01N 33/6896; A61K 2039/505

(57) ABSTRACT

The disclosure pertains to antibodies that bind A-beta oligomers and methods of using said antibodies. Also provided are chimeric or humanized antibodies, including antibodies having specific CDRs identified herein, or a sequence with at least 80% sequence identity to specific VH sequences identified herein, optionally wherein the CDR H3 amino acid sequence is as set forth in any one of SEQ ID NOs: 31-36, 38-40, or 42-50. Also provided are methods and uses thereof as well as kits comprising said antibodies.

### RELATED APPLICATIONS

This is a Patent Cooperation Treaty Application which claims the benefit of 35 U.S.C. § 119 based on the priority of U.S. Provisional Patent Application No. 63/002,899, filed Mar. 31, 2020 which is herein incorporated in its entirety by reference.

### INCORPORATION OF SEQUENCE LISTING

A computer readable form of the Sequence Listing “P60864PC00_ST25_Sequence_Listing” (55,917 bytes), submitted via EFS-WEB and created on Mar. 29, 2021, is herein incorporated by reference.

### FIELD

The present disclosure relates to humanized antibodies that are selective for Amyloid beta (A-beta or Aβ) oligomers as well as compositions and uses thereof.

### BACKGROUND

Amyloid-beta (A-beta), which exists as a 36-43 amino acid peptide, is a product released from amyloid precursor protein (APP) by the enzymes 13 and y secretase. In Alzheimer's disease (AD) patients, A-beta can be present in soluble monomers, insoluble fibrils and soluble oligomers. In monomer form, A-beta exists as a predominantly unstructured polypeptide chain. In fibril form, A-beta can aggregate into distinct morphologies, often referred to as strains. Several of these structures have been determined by solid-state NMR.

Antibodies that bind A-beta have been described.

WO2009048538A2 titled USE OF ANTI-AMYLOID ANTIBODY IN OCULAR DISEASES discloses chimeric antibodies that recognize one or more binding sites on A-beta and are useful for the treatment for ocular diseases.

U.S. Pat. No. 9,221,812B2 titled COMPOUNDS FOR THE TREATMENT OF DISEASES ASSOCIATED WITH AMYLOID OR AMYLOID-LIKE PROTEINS describes pharmaceutical compositions and discontinuous antibodies that bind A-beta including an epitope between amino acid residues 12 to 24 for the treatment of amyloid-related diseases.

WO2003070760A2 titled ANTI-AMYLOID BETA ANTIBODIES AND THEIR USE discloses antibodies that recognize an A-beta discontinuous epitope, wherein the first region comprises the amino acid sequence AEFRHDSGY (SEQ ID NO: 78) or a fragment thereof and wherein the second region comprises the amino acid sequence VHHQKLVFFAEDVG (SEQ ID NO: 79) or a fragment thereof.

US20110171243A1 titled COMPOUNDS TREATING AMYLOIDOSES discloses a peptide mimotope capable of inducing the in vivo formation of antibodies that bind HQKLVF (SEQ ID NO: 80) and/or HQKLVFFAED (SEQ ID NO: 81), and its use.

WO2008088983A1 and WO2001062801A2 disclose a pegylated antibody fragment that binds A-beta amino acids 13-28 and its use in treating A-beta related diseases. Solanezumab and Crenezumab bind amino acids 16-26 on A-beta. Crenezumab interacts with the monomer, oligomer and fibril. Midregion antibodies, including solanezumab (picomolar affinity) and crenezumab (nanomolar affinity), appear to preferentially bind monomeric A-beta (Crespi, G et al, 2015).

WO2009149487A2 titled COMPOUNDS FOR TREATING SYMPTOMS ASSOCIATED WITH PARKINSON'S DISEASE describes compounds comprising a peptide having binding capacity for an antibody specific for an A-beta epitope such as EVHHQKL (SEQ ID NO: 82), HQKLVF (SEQ ID NO: 80) and HQKLVFFAED (SEQ ID NO: 81).

The HHQK (SEQ ID NO: 1) domain is described as involved in plaque induction of neurotoxicity in human microglia, as described in Giulian D et al. 1998 and Winkler et al. 1999. Non-antibody therapeutic agents that bind HHQK (SEQ ID NO: 1) have been disclosed for the treatment of protein folding diseases (US20150105344A1, WO2006125324A1).

U.S. Pat. Nos. 5,766,846; 5,837,672; and 5,593,846 (which are incorporated herein by reference) describe the production of murine monoclonal antibodies to the central domain of the Aβ peptide. WO 01/62801 describes antibodies that bind A-beta between amino acids 13-28. WO2004071408 discloses humanized antibodies.

WO2009086539A2 titled TREATMENT AND PROPHYLAXIS OF AMYLOIDOSIS is directed to Amyloidosis and amyloid light chain amyloidosis, by administering peptides comprising neoepitopes, such as amyloid protein A (AA) fragments from a C-terminal region of AA, and antibodies specific for neoepitopes of aggregated amyloid proteins, for example, antibodies specific for the C-terminal region of AA fibrils.

WO2003070760 titled ANTI-AMYLOID BETA ANTIBODIES AND THEIR USE is directed towards antibody molecules capable of specifically recognizing two regions of the R-A4 peptide, wherein the first region comprises the amino acid sequence AEFRHDSGY (SEQ ID NO: 78) or a fragment thereof and wherein the second region comprises the amino acid sequence VHHAEDVFFAEDVG (SEQ ID NO: 83) or a fragment thereof.

WO2006066089 titled HUMANIZED AMYLOID BETA ANTIBODIES FOR USE IN IMPROVING COGNITION is directed to improved agents and methods for treatment of diseases associated with beta amyloid and in particular to the identification and characterization of a monoclonal antibody, 12A11, that specifically binds to Aβ peptide and is effective at reducing plaque burden associated with amyloidogenic disorders (e.g., AD).

WO2007068429 titled ANTIBODIES AGAINST AMYLOID BETA 4 WITH GLYCOSYLATED IN THE VARIABLE REGION is directed to a purified antibody molecule preparation being characterized in that at least one antigen binding site comprises a glycosylated asparagine (Asn) in the variable region of the heavy chain (V_{H}).

WO 03/016466 is directed variant 266 antibodies that are engineered to lack an N-glycosylation site within the CDR2 of the heavy chain, pharmaceutical compositions thereof, and polynucleotide sequences, vectors, and transformed cells useful to express the variant antibodies. The variants are described to sequester soluble A-beta peptide from human biological fluids and specifically bind an epitope contained within position 13-28 of the amyloid beta peptide.

Yu et al. describes a hexavalent foldable Aβ1-15 (6Aβ15) fused to PADRE or toxin-derived carrier proteins. Wang et al 2016 report that peripheral administration of this antibody mitigates Alzheimer's disease like pathology and cognitive decline in a transgenic animal of aged Alzheimer Disease (Yu Y Z et al. 2014, Wang, H C et al 2016).

WO2017/079833, titled EPITOPES IN AMYLOID BETA MID-REGION AND CONFORMATIONALLY-SELECTIVE ANTIBODIES THERETO, WO 2018/014126, titled ANTIBODIES TO AMYLOID BETA and WO 2019/014768 titled ANTIBODIES TO AMYLOID BETA disclose antibodies that bind the HHQK (SEQ ID NO: 1) epitope in oligomeric A-beta.

### SUMMARY

The present disclosure provides antibodies that preferentially bind oligomeric A-beta. An aspect includes an antibody comprising a light chain variable region and a heavy chain variable region, the heavy chain variable region comprising complementarity determining regions CDR-H1, CDR-H2, and CDR-H3, CDR-H3 comprising a sequence selected from any one of SEQ ID NOs: 31-36, 38-40, 42 or 42-50. Optionally, CDR-H3 comprises a sequence selected from any one of SEQ ID NOs: 31, 33, 34, 38-40, 42, 46-48 or 50. Optionally, CDR-H3 comprises a sequence of SEQ ID NOs: 33 or 42.

In an embodiment, the light chain variable region comprises complementarity determining regions CDR-L1, CDR-L2, and CDR-L3 of SEQ ID NOs: 8, 9, and 10, respectively, CDR-H1 comprises the sequence of SEQ ID NO:5, CDR-H2 comprises the sequence of SEQ ID NO: 6, and CDR3 comprises a sequence selected from any one of SEQ ID NOs: 31-36, 38-40 or 42-50. Optionally, CDR-H3 comprises a sequence selected from any one of SEQ ID NOs: 31, 33, 34, 38-40, 42, 46-48 or 50. Optionally, CDR-H3 comprises a sequence of SEQ ID NOs: 33 or 42.

In an embodiment, the light chain variable region comprises i) an amino acid having the sequence of SEQ ID NO: 4, ii) an amino acid sequence with at least 80%, at least 90%, or at least 95% sequence identity to SEQ ID NO: 4, wherein the CDR-L1, CDR-L2 and CDR-L3 sequences are as set forth in SEQ ID NOs: 8, 9 and 10, respectively, or iii) a conservatively substituted amino acid sequence of i) wherein the CDR-L1, CDR-L2 and CDR-L3 sequences are as set forth in SEQ ID NOs: 8, 9 and 10, respectively.

In an embodiment, the heavy chain variable region comprises i) an amino acid sequence as set forth in any one of SEQ ID NO: 11-16, 18-20, and 22-30, ii) an amino acid sequence with at least 80%, at least 90%, or at least 95% sequence identity to any one of SEQ ID NO: 11-16, 18-20 or 22-30, wherein the CDR-H3 has a sequence as set forth in any one of SEQ ID NOs: 31-36, 38-40 or 42-50, respectively, or iii) a conservatively substituted amino acid sequence of i) wherein the CDR-H3 has a sequence as set forth in any one of SEQ ID NOs: 31-36, 38-40 or 42-50, respectively. Optionally the heavy chain variable region comprises i) an amino acid sequence as set forth in any one of SEQ ID NOs: 13 and 22, ii) an amino acid sequence with at least 80%, at least 90%, or at least 95% sequence identity to SEQ ID NOs: 13 or 22, wherein the CDR-H3 has a sequence as set forth in SEQ ID NOs: 33 or 42, or iii) a conservatively substituted amino acid sequence of i) wherein the CDR-H3 has a sequence as set forth in SEQ ID NOs: 33 or 42.

In an embodiment, the heavy chain variable region amino acid sequence is encoded by a nucleotide sequence as set forth in any one of SEQ ID NOs: 51-56, 58-60 or 62-70; or a codon degenerate or optimized version thereof. In an embodiment, the heavy chain variable region amino acid sequence is encoded by a nucleotide sequence as set forth in any one of SEQ ID NOs: 51, 53, 54, 58-60, 62, 66-68 or 70; or a codon degenerate or optimized version thereof. In an embodiment, the heavy chain variable region amino acid sequence is encoded by a nucleotide sequence as set forth in SEQ ID NOs: 53 or 62; or a codon degenerate or optimized version thereof.

An aspect includes an affinity matured antibody that competes for binding cyclo(CGHHQKG) (SEQ ID NO: 2) peptide and/or oligomeric A-beta with a reference antibody, the reference antibody comprising CDR-H1, CDR-H2, CDRH3, CDR-L1, CDR-L2 and CDR-L3 regions as set forth in SEQ ID NOs: 5 to 10, respectively, or comprising a light chain variable region and a heavy chain variable region as set forth in SEQ ID NOs: 3 and 4, respectively, preferably wherein the antibody has at least 80%, at least 90%, or at least 95% sequence identity to the reference antibody, with the proviso that the affinity matured antibody is not the reference antibody.

In an embodiment, the antibody has greater cyclo(CGHHQKG) (SEQ ID NO: 2) peptide to linear(CGHHQKG) (SEQ ID NO: 2) peptide differential binding activity than the reference antibody by at least or about 3-fold, at least or about 4-fold, at least or about 5-fold, at least or about 6-fold, at least or about 7-fold, at least or about 10-fold, at least or about 15-fold, at least or about 20-fold or at least or about 25-fold.

In an embodiment, the antibody has a KD of at least or about 2.5 10⁻¹¹, at least or about 6×10⁻¹², at least or about 4×10⁻¹², at least or about 2×10⁻¹², at least or about 1×10⁻¹², at least or about 8×10⁻¹³ or at least or about 5×10⁻¹⁵ for a cyclo(CGHHQKG) (SEQ ID NO: 2) peptide.

In an embodiment, the antibody preferentially binds cyclo(CGHHQKG) (SEQ ID NO: 2) peptide over linear(CGHHQKG) (SEQ ID NO: 2) peptide by at least or about 7-fold, at least or about 8-fold, at least or about 9-fold, at least or about 10-fold, at least or about 100-fold, at least or about 200-fold, at least or about 500-fold, at least or about 1000-fold, or at least or about 2000-fold.

In an embodiment, the antibody is an antibody binding fragment selected from Fab, Fab′, F(ab′)2, scFv, dsFv, ds-scFv, dimers, nanobodies, minibodies, diabodies, and multimers thereof.

In an embodiment, the antibody binding fragment is a Fab fragment, optionally comprising the heavy chain variable region of any one of SEQ ID NOs: 11-16, 18-20 or 22-30, preferentially any one of SEQ ID NOs: 11, 13, 14, 18-20, 26-28 or 30, or more preferentially SEQ ID NOs: 13 or 22.

In an embodiment, the antibody comprises the CH1 and/or CL sequence or a part thereof of IgG4, preferably wherein the CH1 and/or CL sequence comprises SEQ ID NOs: 74 and/or 76 or a part thereof, or a conservative variant thereof or a sequence with at least 80%, 90% or 95% sequence identity to SEQ ID NOs: 74 and/or 76.

In an embodiment, the antibody is an IgG1 antibody.

In an embodiment, the antibody is an IgG4 antibody.

In an embodiment, the antibody comprises SEQ ID NOs: 74 and/or 76, and/or CH1 and CH2 of SEQ ID NO: 74 or a conservatively substituted amino acid sequence of any of the foregoing or a sequence with at least 80%, 90% or 95% sequence identity to any of the foregoing.

An aspect includes an antibody that inhibits A-beta oligomer propagation in a subject.

An aspect includes an antibody for treating AD and/or other A-beta amyloid related diseases.

In an embodiment, the antibody is a single chain antibody.

An aspect includes an immunoconjugate comprising the antibody disclosed herein and a detectable label or cytotoxic agent. In an embodiment, the detectable label comprises a positron emitting radionuclide, optionally for use in subject imaging such as PET imaging.

An aspect includes a composition comprising the antibody disclosed herein, or the immunoconjugate disclosed herein, optionally with a diluent.

An aspect includes a nucleic acid molecule encoding the antibody disclosed herein.

An aspect includes a vector comprising the nucleic acid molecule encoding the antibody disclosed herein.

An aspect includes a cell expressing the antibody disclosed herein, optionally wherein the cell is a hybridoma comprising the vector comprising the nucleic acid molecule encoding the antibody.

An aspect includes a kit comprising the antibody disclosed herein, the immunoconjugate disclosed herein, the nucleic acid molecule encoding the antibody disclosed herein, the vector comprising the nucleic acid molecule encoding the antibody disclosed herein, or the cell expressing the antibody disclosed herein.

An aspect includes a method for determining if a biological sample contains A-beta oligomer the method comprising: a) contacting the sample with an antibody disclosed herein or the immunoconjugate disclosed herein that is specific and/or selective for A-beta oligomers under conditions permissive for forming an antibody: A-beta oligomer complex; and b) detecting the presence of any complex; wherein the presence of detectable complex is indicative that the sample may contain or contains A-beta oligomer.

In an embodiment, the amount of complex is measured.

In an embodiment, the sample comprises brain tissue or an extract thereof, whole blood, plasma, serum and/or CSF.

In an embodiment, the sample is compared to a control, optionally a previous sample.

An aspect includes a method of measuring a level of oligomeric A-beta in a subject, the method comprising administering to a subject at risk or suspected of having or having AD an immunoconjugate comprising the antibody disclosed herein conjugated to a detectable label; and detecting the label, optionally quantitatively detecting the label.

In an embodiment, the label is a positron emitting radionuclide.

An aspect includes a method of inhibiting A-beta oligomer propagation, the method comprising contacting a cell or tissue expressing A-beta with or administering to a subject in need thereof an effective amount of an A-beta oligomer specific or selective antibody disclosed herein or immunoconjugate disclosed herein to inhibit A-beta aggregation and/or oligomer propagation.

An aspect includes a method of treating AD and/or other A-beta amyloid related diseases, the method comprising administering to a subject in need thereof i) an effective amount of an antibody disclosed herein or immunoconjugate disclosed herein, or a pharmaceutical composition comprising said antibody or said immunoconjugate; or 2) a nucleic acid or vector comprising a nucleic acid encoding said antibody, to a subject in need thereof.

In an embodiment, a biological sample from the subject to be treated is assessed for the presence or levels of A-beta using an antibody described herein.

In an embodiment, the antibody, immunoconjugate, composition or nucleic acid or vector is administered directly to the brain or other portion of the CNS.

In an embodiment, the composition is a pharmaceutical composition comprising the antibody or immunoconjugate in admixture with a pharmaceutically acceptable, diluent or carrier.

Other features and advantages of the present disclosure will become apparent from the following detailed description. It should be understood, however, that the detailed description and the specific examples while indicating preferred embodiments of the disclosure are given by way of illustration only, since various changes and modifications within the spirit and scope of the disclosure will become apparent to those skilled in the art from this detailed description.

### BRIEF DESCRIPTION OF THE DRAWINGS

An embodiment of the present disclosure will now be described in relation to the drawings in which:

FIGS. 1A and 1B are representative bar graphs showing results of ELISA binding assays against cyclo-BSA peptide (FIG. 1A) and linear-BSA peptide (FIG. 1B) for selected clones.

FIG. 2 is a series of graphs showing surface plasmon resonance (SPR) sensorgrams for selected antibody clones binding to cyclo-BSA peptide and linear-BSA peptide.

FIGS. 3A and 3B show multi-cycle kinetics sensorgrams and fitted curves of the purified lead and parental antibodies to cyclic peptide-BSA (FIG. 3A); and linear peptide-BSA (FIG. 3B).

### DETAILED DESCRIPTION OF THE DISCLOSURE

Provided herein are antibodies comprising a CDR3 sequence selected from those as shown in Table 4. Also provided in an embodiment are antibodies having the variable region sequences provided in Table 3, as well as immunotherapeutic compositions thereof and methods of use thereof. Said antibodies may target epitopes preferentially accessible in toxic oligomeric species of A-beta, including oligomeric species associated with Alzheimer's disease (AD).

As shown herein and in WO 2019/014768, an antibody that binds a cyclic peptide comprising the A-beta epitope HHQK (SEQ ID NO: 1), having the CDRs indicated, preferentially bound oligomeric Abeta and selectively bound the cyclic peptide compared to a linear peptide of the same sequence (i.e., corresponding linear sequence).

As shown in the Examples described herein, antibodies showed one or more improved characteristics such as improved binding of oligomeric A-beta and/or improved specificity for conformational cyclic peptide vs linear peptide compared to the parental antibody.

Accordingly, in some embodiments the antibody has increased binding, such as 2-fold increased or up to 2000-fold increased (or any number in between) compared to parental or reference antibody, to cyclic peptide and/or equal or decreased binding compared to parental or reference antibody, to linear peptide.

In some embodiments the antibody has decreased binding, such as lacking appreciable binding compared to parental antibody, to linear peptide, and/or equal or increased binding compared to parental antibody, to cyclic peptide.

In some embodiments, the antibody has an increased binding specificity for cyclic vs linear peptide compared to parental antibody.

In some embodiments the antibody has 7-fold greater specificity, 8-fold greater specificity, 9-fold greater specificity, 10-fold greater specificity, 100-fold greater specificity, 200-fold greater specificity, 500-fold greater specificity, 1000-fold greater specificity, or more than 2000-fold greater specificity (or any number in between) for cyclic vs linear peptide.

### I. Definitions

As used herein, the term ‘A-beta’ may alternately be referred to as ‘amyloid beta’, ‘amyloid 13’, A-beta, A-beta or ‘A13’. Amyloid beta is a peptide of 36-43 amino acids and includes all wild-type and mutant forms of all species, particularly human A-beta. A-beta40 refers to the 40 amino acid form; A-beta42 refers to the 42 amino acid form, etc. The amino acid sequence of human wild-type A-beta42 is shown in SEQ ID NO: 77.

As used herein, the term “A-beta monomer” herein refers to any of the individual subunit forms of the A-beta (e.g., 1-40, 1-42, 1-43) peptide.

As used herein, the term “A-beta oligomer” herein refers to a plurality of any of the A-beta subunits wherein several (e.g., at least two) A-beta monomers are non-covalently aggregated in a conformationally-flexible, partially-ordered, three-dimensional globule of less than about 100, or more typically less than about 50 monomers. For example, an oligomer may contain 3 or 4 or 5 or more monomers. The term “A-beta oligomer” as used herein includes both synthetic A-beta oligomer and/or native A-beta oligomer. “Native A-beta oligomer” refers to A-beta oligomer formed in vivo, for example in the brain and CSF of a subject with AD.

As used herein, the term “A-beta fibril” refers to a molecular structure that comprises assemblies of non-covalently associated, individual A-beta peptides which show fibrillary structure under an electron microscope. The fibrillary structure is typically a “cross beta” structure; there is no theoretical upper limit on the size of multimers, and fibrils may comprise thousands or many thousands of monomers. Fibrils can aggregate by the thousands to form senile plaques, one of the primary pathological morphologies diagnostic of AD.

The term “HHQK” means the amino acid sequence histidine, histidine, glutamine, lysine, as shown in SEQ ID NO: 1. Depending on the context, the reference of the amino acid sequence can refer to a sequence in A-beta or an isolated peptide, such as the amino acid sequence of a cyclic compound.

The term “amino acid” includes all of the naturally occurring amino acids as well as modified L-amino acids. The atoms of the amino acid can include different isotopes. For example, the amino acids can comprise deuterium substituted for hydrogen nitrogen-15 substituted for nitrogen-14, and carbon-13 substituted for carbon-12 and other similar changes.

The term “antibody” as used herein is intended to include monoclonal antibodies, polyclonal antibodies, single chain, veneered, humanized and other chimeric antibodies and binding fragments thereof, including for example a single chain Fab fragment, Fab′2 fragment or single chain Fv fragment. The antibody may be from recombinant sources and/or produced in animals such as rabbits, llamas, sharks etc. Also included are human antibodies that can be produced in transgenic animals or using biochemical techniques or can be isolated from a library such as a phage library. Humanized or other chimeric antibodies may include sequences from one or more than one isotype or class or species. Antibodies may be any class of immunoglobulins including: IgM, IgG, IgD, IgA or IgE; and any isotype, including: IgG1, IgG2 (e.g. IgG2a, IgG2b), IgG3 and IgG4. The antibody may include sequences from one or more than one isotype or class. Further, these antibodies are typically produced as antigen binding fragments such as Fab, Fab′ F(ab′)2, Fd, Fv and single domain antibody fragments, or as single chain antibodies in which the heavy and light chains are linked by a spacer. Also, the antibodies may exist in monomeric or polymeric form. The antibody optionally comprises one non-human chain and one humanized chain (i.e., one humanized heavy or light chain).

The term “parental antibody” as used herein is intended to refer to an antibody having the CDR sequences set out in Table 2. The parental antibody can for example be a humanized antibody having a heavy chain variable sequence set out in SEQ ID NO: 3, a light chain variable sequence set out in SEQ ID NO:4, and humanized IgG4 sequences set out in SEQ ID NO: 74 and SEQ ID NO: 76.

The phrase “isolated antibody” refers to antibody produced in vivo or in vitro that has been removed from the source that produced the antibody, for example, an animal, hybridoma or other cell line (such as recombinant insect, yeast or bacteria cells that produce antibody). The isolated antibody is optionally “purified”, which means at least: 80%, 85%, 90%, 95%, 98% or 99% purity.

The term “binding fragment” as used herein to a part or portion of an antibody or antibody chain comprising fewer amino acid residues than an intact or complete antibody or antibody chain and which binds the antigen or competes with intact antibody. Exemplary binding fragments include without limitations Fab, Fab′, F(ab′)2, scFv, dsFv, ds-scFv, dimers, nanobodies, minibodies, diabodies, and multimers thereof. Fragments can be obtained via chemical or enzymatic treatment of an intact or complete antibody or antibody chain. Fragments can also be obtained by recombinant means. For example, F(ab′)2 fragments can be generated by treating the antibody with pepsin. The resulting F(ab′)2 fragment can be treated to reduce disulfide bridges to produce Fab′ fragments. Papain digestion can lead to the formation of Fab fragments. Fab, Fab′ and F(ab′)2, scFv, dsFv, ds-scFv, dimers, minibodies, diabodies, bispecific antibody fragments and other fragments can also be constructed by recombinant expression techniques.

The term “complementarity determining region” or “CDR” as used herein refers to particular hypervariable regions of antibodies that are commonly understood to define epitope binding. Computational methods for defining CDR sequences include for example IMGT, Kabat and Chothia numbering schemes. Unless specified otherwise, the CDRs listed in the present disclosure are defined using IMGT numbering. A person skilled in the art having regard to the sequences comprised herein would also be able to identify CDR sequences based on Kabat and Chothia etc. It will also be understood that the CDR sequences may vary depending on the computational method chosen. For example, CDR sequences of the parental antibody, as defined by IMGT and Kabat, are provided in Table 2, and CDR sequences of the presently disclosed antibodies, as defined by IMGT and Kabat, are provided in Table 4.

The terms “IMGT numbering” or “ImMunoGeneTics database numbering”, which are recognized in the art, refer to a system of numbering amino acid residues which are more variable (i.e., hypervariable) than other amino acid residues in the heavy and light chain variable regions of an antibody, or antigen binding portion thereof.

As used herein, the term “conformational epitope” refers to an epitope where the epitope amino acid sequence has a particular three-dimensional structure wherein at least an aspect of the three-dimensional structure not present or less likely to be present in another form for example a corresponding linear peptide or Abeta monomer and is specifically and/or selectively recognized by the cognate antibody. Antibodies which specifically bind a conformation-specific epitope recognize the spatial arrangement of one or more of the amino acids of that conformation-specific epitope. For example, an HHQK (SEQ ID NO: 1) conformational epitope refers to an epitope of HHQK (SEQ ID NO: 1) that is recognized by antibodies selectively, for example at least 2 fold, 3 fold, 5 fold, 10 fold, 50 fold, 100 fold, 250 fold, 500 fold or 1000 fold or greater more selectivity as compared to antibodies raised using linear HHQK (SEQ ID NO: 1). When an antibody is said to selectively bind an epitope such as a conformational epitope, such as HHQK (SEQ ID NO: 1), what is meant is that the antibody preferentially binds one or more particular conformations containing the specified residues or a part thereof with greater affinity than it binds said residues in another conformation. For example, when an antibody is said to selectively bind a cyclopeptide comprising HHQK (SEQ ID NO: 1) or related epitope relative to a corresponding linear peptide, the antibody binds the cyclopeptide with at least a 2 fold greater affinity than it binds the linear peptide. Similarly, when an antibody is said to selectively bind oligomeric Abeta, the antibody binds the oligomeric species with at least a 2 fold greater affinity than it binds Abeta monomer and/or plaque fibrils.

The term “lacks or has negligible plaque binding” as used herein with respect to an antibody means that the antibody does not show typical plaque morphology staining on immunohistochemistry (e.g., in situ, optionally as compared to plaque staining seen with Abeta antibody 6E10) and the level of staining is comparable to or no more than 2 fold the level seen with an IgG negative (e.g. irrelevant) isotype control.

The term “isolated peptide” refers to peptide that has been produced, for example, by recombinant or synthetic techniques, and removed from the source that produced the peptide, such as recombinant cells or residual peptide synthesis reactants. The isolated peptide is optionally “purified”, which means at least: 80%, 85%, 90%, 95%, 98% or 99% purity and optionally pharmaceutical grade purity.

The term “detectable label” as used herein refers to moieties such as peptide sequences (such a myc tag, HA-tag, V5-tag or NE-tag), fluorescent proteins that can be appended or introduced into a peptide or compound described herein and which is capable of producing, either directly or indirectly, a detectable signal. For example, the label may be radio-opaque, positron-emitting radionuclide (for example for use in PET imaging), or a radioisotope, such as ³H, ¹³N, ¹⁴C, ¹⁸F, ³²P, ³⁵S, ¹²³I, ¹²⁵I ¹³¹I; a fluorescent (fluorophore) or chemiluminescent (chromophore) compound, such as fluorescein isothiocyanate, rhodamine or luciferin; an enzyme, such as alkaline phosphatase, beta-galactosidase or horseradish peroxidase; an imaging agent; or a metal ion. The detectable label may be also detectable indirectly for example using secondary antibody.

The term “epitope” as commonly used means an antibody binding site, typically a polypeptide segment, in an antigen that is specifically recognized by the antibody. As used herein “epitope” can also refer to the amino acid sequences or part thereof identified on A-beta using the collective coordinates method described. For example an antibody generated against an isolated peptide corresponding to a cyclic compound comprising the identified target region HHQK (SEQ ID NO: 1), recognizes part or all of said epitope sequence. An epitope is “accessible” in the context of the present specification when it is accessible to binding by an antibody.

The term “greater affinity” as used herein refers to a relative degree of antibody binding where an antibody X binds to target Y more strongly (Kₒₙ) and/or with a smaller dissociation constant (K_{off}) than to target Z, and in this context antibody X has a greater affinity for target Y than for Z. Likewise, the term “lesser affinity” herein refers to a degree of antibody binding where an antibody X binds to target Y less strongly and/or with a larger dissociation constant than to target Z, and in this context antibody X has a lesser affinity for target Y than for Z. The affinity of binding between an antibody and its target antigen, can be expressed as KA equal to 1/K_{D} where K_{D} is equal to kₒₙ/k_{off}. As such, a greater affinity corresponds to a lower K_{D}. The kₒₙ and k_{off} values can be measured using surface plasmon resonance technology, for example using a Molecular Affinity Screening System (MASS-1) (Sierra Sensors GmbH, Hamburg, Germany). An antibody that is selective for a conformation presented in a cyclic compound optional a cyclic peptide for example has a greater affinity for the cyclic compound (e.g. cyclic peptide) compared to a corresponding sequence in linear form (e.g. the sequence non-cyclized).

The term “corresponding linear compound” with regard to a cyclic compound refers to a compound, optionally a peptide, comprising or consisting of the same sequence or chemical moieties as the cyclic compound but in linear (i.e. non-cyclized) form, for example having properties as would be present in solution of a linear peptide. For example, the corresponding linear compound can be the synthesized peptide that is not cyclized.

As used herein “specifically binds” in reference to an antibody means that the antibody recognizes an epitope sequence and binds to its target antigen with a minimum affinity. For example a multivalent antibody binds its target with a K_{D} of at least 1e-6, at least 1e-7, at least 1e-8, at least 1e-9, or at least 1e-10. Affinities greater than at least 1e-8 may be preferred. For example the K_{D} may be in the nanomolar range or the picomolar range. An antigen binding fragment such as Fab fragment comprising one variable domain, may bind its target with a 10 fold or 100 fold less affinity than a multivalent interaction with a non-fragmented antibody.

The term “selectively binds” as used herein with respect to an antibody that selectively binds a form of A-beta (e.g. fibril, monomer or oligomer) or a cyclic compound means that the antibody binds the form with at least 2 fold, at least 3 fold, or at least 5 fold, at least 10 fold, at least 100 fold, at least 250 fold, at least 500 fold or at least 1000 fold or more greater affinity. Accordingly an antibody that is more selective for a particular conformation (e.g. oligomer) preferentially binds the particular form of A-beta with at least 2 fold etc. greater affinity compared to another form and/or a linear peptide.

The term “animal” or “subject” as used herein includes all members of the animal kingdom including mammals, optionally including or excluding humans.

A “conservative amino acid substitution” as used herein, is one in which one amino acid residue is replaced with another amino acid residue without abolishing the protein's desired properties. Suitable conservative amino acid substitutions can be made by substituting amino acids with similar hydrophobicity, polarity, and R-chain length for one another. Examples of conservative amino acid substitution include:

Conservative Substitutions

| Type of Amino Acid | Substitutable Amino Acids |
|---|---|
| Hydrophilic | Ala, Pro, Gly, Glu, Asp, Gln, Asn, Ser, Thr |
| Sulphydryl | Cys |
| Aliphatic | Val, Ile, Leu, Met |
| Basic | Lys, Arg, His |
| Aromatic | Phe, Tyr, Trp |

The term “sequence identity” as used herein refers to the percentage of sequence identity between two polypeptide sequences or two nucleic acid sequences. To determine the percent identity of two amino acid sequences or of two nucleic acid sequences, the sequences are aligned for optimal comparison purposes (e.g., gaps can be introduced in the sequence of a first amino acid or nucleic acid sequence for optimal alignment with a second amino acid or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences (i.e., % identity=number of identical overlapping positions/total number of positions.times.100%). In one embodiment, the two sequences are the same length. The determination of percent identity between two sequences can also be accomplished using a mathematical algorithm. A preferred, non-limiting example of a mathematical algorithm utilized for the comparison of two sequences is the algorithm of Karlin and Altschul, 1990, Proc. Natl. Acad. Sci. U.S.A. 87:2264-2268, modified as in Karlin and Altschul, 1993, Proc. Natl. Acad. Sci. U.S.A. 90:5873-5877. Such an algorithm is incorporated into the NBLAST and XBLAST programs of Altschul et al., 1990, J. Mol. Biol. 215:403. BLAST nucleotide searches can be performed with the NBLAST nucleotide program parameters set, e.g., for score=100, word length=12 to obtain nucleotide sequences homologous to a nucleic acid molecules of the present application. BLAST protein searches can be performed with the XBLAST program parameters set, e.g., to score-50, word length=3 to obtain amino acid sequences homologous to a protein molecule described herein. To obtain gapped alignments for comparison purposes, Gapped BLAST can be utilized as described in Altschul et al., 1997, Nucleic Acids Res. 25:3389-3402. Alternatively, PSI-BLAST can be used to perform an iterated search which detects distant relationships between molecules (Id.). When utilizing BLAST, Gapped BLAST, and PSI-Blast programs, the default parameters of the respective programs (e.g., of XBLAST and NBLAST) can be used (see, e.g., the NCBI website). Another preferred non-limiting example of a mathematical algorithm utilized for the comparison of sequences is the algorithm of Myers and Miller, 1988, CABIOS 4:11-17. Such an algorithm is incorporated in the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used. The percent identity between two sequences can be determined using techniques similar to those described above, with or without allowing gaps. In calculating percent identity, typically only exact matches are counted.

For antibodies, percentage sequence identities can be determined when antibody sequences maximally aligned by IMGT or other (e.g. Kabat or Chothia numbering scheme). After alignment, if a subject antibody region (e.g., the entire mature variable region of a heavy or light chain) is being compared with the same region of a reference antibody, the percentage sequence identity between the subject and reference antibody regions is the number of positions occupied by the same amino acid in both the subject and reference antibody region divided by the total number of aligned positions of the two regions, with gaps not counted, multiplied by 100 to convert to percentage.

The term “nucleic acid sequence” as used herein refers to a sequence of nucleoside or nucleotide monomers consisting of naturally occurring bases, sugars and intersugar (backbone) linkages. The term also includes modified or substituted sequences comprising non-naturally occurring monomers or portions thereof. The nucleic acid sequences of the present application may be deoxyribonucleic acid sequences (DNA) or ribonucleic acid sequences (RNA) and may include naturally occurring bases including adenine, guanine, cytosine, thymidine and uracil. The sequences may also contain modified bases. Examples of such modified bases include aza and deaza adenine, guanine, cytosine, thymidine and uracil; and xanthine and hypoxanthine. The nucleic acid can be either double stranded or single stranded, and represents the sense or antisense strand. Further, the term “nucleic acid” includes the complementary nucleic acid sequences as well as codon optimized or synonymous codon equivalents. The term “isolated nucleic acid sequences” as used herein refers to a nucleic acid substantially free of cellular material or culture medium when produced by recombinant DNA techniques, or chemical precursors, or other chemicals when chemically synthesized. An isolated nucleic acid is also substantially free of sequences which naturally flank the nucleic acid (i.e. sequences located at the 5′ and 3′ ends of the nucleic acid) from which the nucleic acid is derived.

“Operatively linked” is intended to mean that the nucleic acid is linked to regulatory sequences in a manner which allows expression of the nucleic acid. Suitable regulatory sequences may be derived from a variety of sources, including bacterial, fungal, viral, mammalian, or insect genes. Selection of appropriate regulatory sequences is dependent on the host cell chosen and may be readily accomplished by one of ordinary skill in the art. Examples of such regulatory sequences include: a transcriptional promoter and enhancer or RNA polymerase binding sequence, a ribosomal binding sequence, including a translation initiation signal. Additionally, depending on the host cell chosen and the vector employed, other sequences, such as an origin of replication, additional DNA restriction sites, enhancers, and sequences conferring inducibility of transcription may be incorporated into the expression vector.

The term “vector” as used herein comprises any intermediary vehicle for a nucleic acid molecule which enables said nucleic acid molecule, for example, to be introduced into prokaryotic and/or eukaryotic cells and/or integrated into a genome, and include plasmids, phagemids, bacteriophages or viral vectors such as retroviral based vectors, Adeno Associated viral vectors and the like. The term “plasmid” as used herein generally refers to a construct of extrachromosomal genetic material, usually a circular DNA duplex, which can replicate independently of chromosomal DNA.

By “at least moderately stringent hybridization conditions” it is meant that conditions are selected which promote selective hybridization between two complementary nucleic acid molecules in solution. Hybridization may occur to all or a portion of a nucleic acid sequence molecule. The hybridizing portion is typically at least 15 (e.g. 20, 25, 30, 40 or 50) nucleotides in length. Those skilled in the art will recognize that the stability of a nucleic acid duplex, or hybrids, is determined by the Tm, which in sodium containing buffers is a function of the sodium ion concentration and temperature (Tm=81.5° C.−16.6 (Log 10 [Na+])+0.41(%(G+C)−600/I), or similar equation). Accordingly, the parameters in the wash conditions that determine hybrid stability are sodium ion concentration and temperature. In order to identify molecules that are similar, but not identical, to a known nucleic acid molecule a 1% mismatch may be assumed to result in about a 1° C. decrease in Tm, for example if nucleic acid molecules are sought that have a >95% identity, the final wash temperature will be reduced by about 5° C. Based on these considerations those skilled in the art will be able to readily select appropriate hybridization conditions. In preferred embodiments, stringent hybridization conditions are selected. By way of example the following conditions may be employed to achieve stringent hybridization: hybridization at 5× sodium chloride/sodium citrate (SSC)/5×Denhardt's solution/1.0% SDS at Tm—5° C. based on the above equation, followed by a wash of 0.2×SSC/0.1% SDS at 60° C. Moderately stringent hybridization conditions include a washing step in 3×SSC at 42° C. It is understood, however, that equivalent stringencies may be achieved using alternative buffers, salts and temperatures. Additional guidance regarding hybridization conditions may be found in: Current Protocols in Molecular Biology, John Wiley & Sons, N.Y., 2002, and in: Sambrook et al., Molecular Cloning: a Laboratory Manual, Cold Spring Harbor Laboratory Press, 2001.

The term “treating” or “treatment” as used herein and as is well understood in the art, means an approach for obtaining beneficial or desired results, including clinical results. Beneficial or desired clinical results can include, but are not limited to, alleviation or amelioration of one or more symptoms or conditions, diminishment of extent of disease, stabilized (i.e. not worsening) state of disease, preventing spread of disease, delay or slowing of disease progression, amelioration or palliation of the disease state, diminishment of the reoccurrence of disease, and remission (whether partial or total), whether detectable or undetectable. “Treating” and “Treatment” can also mean prolonging survival as compared to expected survival if not receiving treatment. “Treating” and “treatment” as used herein also include prophylactic treatment. For example, a subject with early stage AD can be treated to prevent progression can be treated with a compound, antibody, immunogen, nucleic acid or composition described herein to prevent progression.

The term “administered” as used herein means administration of a therapeutically effective dose of a compound or composition of the disclosure to a cell or subject.

As used herein, the phrase “effective amount” means an amount effective, at dosages and for periods of time necessary to achieve a desired result. Effective amounts when administered to a subject may vary according to factors such as the disease state, age, sex, weight of the subject. Dosage regime may be adjusted to provide the optimum therapeutic response.

The term “pharmaceutically acceptable” means that the carrier, diluent, or excipient is compatible with the other components of the formulation and not substantially deleterious to the recipient thereof.

Compositions or methods “comprising” or “including” one or more recited elements may include other elements not specifically recited. For example, a composition that “comprises” or “includes” an antibody may contain the antibody alone or in combination with other ingredients.

In understanding the scope of the present disclosure, the term “consisting” and its derivatives, as used herein, are intended to be close ended terms that specify the presence of stated features, elements, components, groups, integers, and/or steps, and also exclude the presence of other unstated features, elements, components, groups, integers and/or steps.

The recitation of numerical ranges by endpoints herein includes all numbers and fractions subsumed within that range (e.g. 1 to 5 includes 1, 1.5, 2, 2.75, 3, 3.90, 4, and 5). It is also to be understood that all numbers and fractions thereof are presumed to be modified by the term “about.” Further, it is to be understood that “a,” “an,” and the include plural referents unless the content clearly dictates otherwise. The term “about” means plus or minus 0.1 to 50%, 5-50%, or 10-40%, preferably 10-20%, more preferably 10% or 15%, of the number to which reference is being made.

Further, the definitions and embodiments described in particular sections are intended to be applicable to other embodiments herein described for which they are suitable as would be understood by a person skilled in the art. For example, in the following passages, different aspects of the invention are defined in more detail. Each aspect so defined may be combined with any other aspect or aspects unless clearly indicated to the contrary. In particular, any feature indicated as being preferred or advantageous may be combined with any other feature or features indicated as being preferred or advantageous.

The singular forms of the articles “a,” “an,” and “the” include plural references unless the context clearly dictates otherwise. For example, the term “a compound” or “at least one compound” can include a plurality of compounds, including mixtures thereof.

### II. Antibodies and Nucleic Acids

Disclosed herein are antibodies that have one or more improved features and uses thereof.

As demonstrated in the Examples, antibodies were sequenced, selectively bound the cyclic compound cyclo(CGHHQKG) (SEQ ID NO: 2) relative to the corresponding linear peptide, selectively bound A-beta oligomer over monomer, and/or lacked appreciable plaque staining in AD tissue.

Accordingly, an aspect includes an antibody comprising a light chain variable region and a heavy chain variable region, the heavy chain variable region comprising complementarity determining regions CDR-H1, CDR-H2, and CDR-H3, the CDR-H3 having a sequence of any one of SEQ ID NOs: 31-36, 38-40 or 42-50. In an embodiment, CDR-H1 has the sequence of SEQ ID NO: 5, CDR-H2 has the sequence of SEQ ID NO: 6, and/or the light chain variable region comprises complementarity determining regions CDR-L1, CDR-L2, and CDR-L3 as set forth in SEQ ID NOs: 8, 9, and 10, respectively.

Another aspect includes an antibody that competes for binding cyclo(CGHHQKG) (SEQ ID NO: 2) peptide and/or oligomeric A-beta with a reference antibody, the reference antibody comprising CDR-H1, CDR-H2, CDRH3, CDR-L1, CDR-L2 and CDR-L3 regions as set forth in SEQ ID NOs: 5 to 10, respectively, or comprising a light chain variable region and a heavy chain variable region as set forth in SEQ ID NOs: 3 and 4, respectively, preferably wherein the antibody has at least 80%, at least 90%, or at least 95% sequence identity to the reference antibody.

Competition between antibodies can be determined for example using an assay in which an antibody under test is assessed for its ability to inhibit specific binding of a reference antibody to the common antigen. A test antibody competes with a reference antibody if the test antibody (e.g. equal amount or in excess of at least a 2 fold, 5, fold, 10 fold or 20 fold) inhibits binding of the reference antibody by at least 50%, at least 75%, at least 80%, at least 90% or at least 95% as measured in a competitive binding assay. Competitive binding assays can include for example surface plasmon resonance (SPR) (for example using a Molecular Affinity Screening System (MASS-1) or a Biacore™ SPR System), enzyme-linked immunosorbent assay (ELISA), electro-chemiluminescence binding assay (ECLIA) or other assay where binding of the reference antibody to its target is measured in the presence or absence of different concentrations of the test antibody. It will be understood that SPR binding response (e.g. BRU-binding response units) can be influenced by several factors including the particular conditions of the assay, the equipment used and other experimental conditions that may influence the read-out, such as density of antibody on the chip, concentration of peptide injected, flow rate, buffer composition, etc.

In an embodiment, the antibody does not bind monomeric A-beta, for example under conditions described in the Examples. In an embodiment, the antibody does not bind A-beta in senile plaques, for example in situ in AD brain tissue, for example under conditions described in the Examples.

In another embodiment, the antibody does not selectively bind monomeric A-beta compared to native- or synthetic-oligomeric A-beta.

In an embodiment, the A-beta oligomer comprises A-beta 1-42 subunits.

In an embodiment, the antibody lacks A-beta fibril plaque (also referred to as senile plaque) staining, for example as measured by immunohistochemistry. Absence of plaque staining can be assessed by comparing to a positive control such as pan A-beta-specific antibodies 6E10 and 4G8 (Biolegend, San Diego, Calif.), or 2C8 (Enzo Life Sciences Inc., Farmingdale, N.Y.) and an isotype control. An antibody described herein lacks or has negligible A-beta fibril plaque staining if the antibody does not show typical plaque morphology staining and the level of staining is comparable to or no more than 2 fold the level seen with an IgG negative isotype control. The scale can for example set the level of staining with isotype control at 1 and with 6E10 at 10. An antibody lacks A-beta fibril plaque staining if the level of staining on such a scale is 2 or less. In embodiment, the antibody shows minimal A-beta fibril plaque staining, for example on the foregoing scale, levels scored at less about or less than 3.

A further aspect is an antibody conjugated to a therapeutic, detectable label or cytotoxic agent. In an embodiment, the detectable label is a positron-emitting radionuclide. A positron-emitting radionuclide can be used for example in PET imaging.

A further aspect relates to an antibody complex comprising an antibody described herein and/or a binding fragment thereof and oligomeric A-beta.

A further aspect is an isolated nucleic acid encoding an antibody or part thereof described herein.

Nucleic acids encoding a heavy chain or a light chain or parts thereof are also provided, for example encoding a heavy chain comprising CDR-H1, CDR-H2 and/or CDR-H3 regions described herein or encoding a light chain comprising CDR-L1, CDR-L2 and/or CDR-L3 regions described herein, variable chains described herein and codon optimized and codon degenerate versions thereof.

The present disclosure also provides variants of the nucleic acid sequences that encode for the antibody and/or binding fragment thereof disclosed herein. For example, the variants include nucleotide sequences that hybridize to the nucleic acid sequences encoding the antibody and/or binding fragment thereof disclosed herein under at least moderately stringent hybridization conditions or codon degenerate or optimized sequences.

A further aspect is an isolated nucleic acid encoding an antibody described herein.

Another aspect is an expression cassette or vector comprising the nucleic acid herein disclosed. In an embodiment, the vector is an isolated vector.

The vector can be any vector, including vectors suitable for producing an antibody and/or binding fragment thereof or expressing a peptide sequence described herein.

The nucleic acid molecules may be incorporated in a known manner into an appropriate expression vector which ensures expression of the protein. Possible expression vectors include but are not limited to cosmids, plasmids, or modified viruses (e.g. replication defective retroviruses, adenoviruses and adeno-associated viruses). The vector should be compatible with the host cell used. The expression vectors are “suitable for transformation of a host cell”, which means that the expression vectors contain a nucleic acid molecule encoding the peptides corresponding to epitopes or antibodies described herein.

In an embodiment, the vector is suitable for expressing for example single chain antibodies by gene therapy. The vector can be adapted for specific expression in neural tissue, for example using neural specific promoters and the like. In an embodiment, the vector comprises an IRES and allows for expression of a light chain variable region and a heavy chain variable region. Such vectors can be used to deliver antibody in vivo.

Suitable regulatory sequences may be derived from a variety of sources, including bacterial, fungal, viral, mammalian, or insect genes.

Examples of such regulatory sequences include: a transcriptional promoter and enhancer or RNA polymerase binding sequence, a ribosomal binding sequence, including a translation initiation signal. Additionally, depending on the host cell chosen and the vector employed, other sequences, such as an origin of replication, additional DNA restriction sites, enhancers, and sequences conferring inducibility of transcription may be incorporated into the expression vector.

In an embodiment, the regulatory sequences direct or increase expression in neural tissue and/or cells.

In an embodiment, the vector is a viral vector.

In one embodiment, the vector expresses a single chain antibody herein disclosed.

Vectors comprising single chain antibodies can be prepared by several methods.

For example, scFv nucleic acids can be constructed in various formats, such as in a FLAG tag-VH-linker-VL-Lyslinker-lysosomal targeting sequence format. An antibody heavy chain variable domain (VH) and light chain variable domain (VL) can each consist of FR1-CDR1-FR2-CDR2-FR3-CDR3-FR4. Linkers for linking the VH and VL sequences and for linking the lysosomal linker can consist of 3 and 1 tandem repeats of GGGGS, respectively. The scFv nucleic acid, flanked by 5′-Nhel/Hindlll-3′ restriction sequences, can be synthesized by sequentially linking individual phosphoramidite monomers using solid-phase phosphoramidite chemistry methods on a DNA synthesizer. The resulting single-stranded scFv gene insert can be amplified by standard PCR to generate double-stranded insert, which can then be cloned into a Nhel/Hindlll restriction enzyme digested pcDNA3.1(−) vector.

Other methods can also be used. For example mRNA can be isolated from a hybridoma cell line using a mRNA isolation kit (Qiagen, Chatsworth, Calif.). cDNA can be synthesized using for example Superscript First Strand, catalog no. 12371-019 (Invitrogen, Carlsbad, Calif.) with oligodT priming according to the manufacturer's instructions. The variable regions of heavy chain (VH) and light chain (V_{K}) can amplified separately from first-strand cDNA by using a mixture of universal polymerase chain reaction (PCR) primers and Platinum Pfx DNA polymerase (Invitrogen). The PCR products for heavy chain and light chain can be cut with restriction enzymes such as Pstl/BstEll and Sacl/Xhol, respectively and agarose gel-purified. The cDNA inserts corresponding to VL and VH can be cloned into for example pBZUT7 vector and sequenced. The VH and VL domains can be assembled and linked together by PCR to yield the full-length scFv nucleic acid. The scFv nucleic acid can then be subcloned upstream of a Myc-tag. The construct can also be designed to include one or more moieties such as a signal sequence for efficient secretion, a tag such as c-myc epitope or FLAG to facilitate detection or a targeting moiety such as a lysosomal signal sequence or an autophagy signal sequence.

The recombinant expression vectors may also contain a marker gene which facilitates the selection of host cells transformed, infected or transfected with a vector for expressing an antibody or epitope peptide described herein.

The recombinant expression vectors may also contain expression cassettes which encode a fusion moiety (i.e. a “fusion protein”) which provides increased expression or stability of the recombinant peptide; increased solubility of the recombinant peptide; and aid in the purification of the target recombinant peptide by acting as a ligand in affinity purification, including for example tags and labels described herein. Further, a proteolytic cleavage site may be added to the target recombinant protein to allow separation of the recombinant protein from the fusion moiety subsequent to purification of the fusion protein. Typical fusion expression vectors include pGEX (Amrad Corp., Melbourne, Australia), pMAL (New England Biolabs, Beverly, Mass.) and pRIT5 (Pharmacia, Piscataway, N.J.) which fuse glutathione S-transferase (GST), maltose E binding protein, or protein A, respectively, to the recombinant protein.

Systems for the transfer of genes for example into neurons and neural tissue both in vitro and in vivo include vectors based on viruses, most notably Herpes Simplex Virus, Adenovirus, Adeno-associated virus (AAV) and retroviruses including lentiviruses. Alternative approaches for gene delivery include the use of naked, plasmid DNA as well as liposome-DNA complexes. Another approach is the use of AAV plasmids in which the DNA is polycation-condensed and lipid entrapped and introduced into the brain by intracerebral gene delivery (Leone et al. US Application No. 2002076394).

Accordingly, in another aspect, the compounds, immunogens, nucleic acids, vectors and antibodies described herein may be formulated in vesicles such as liposomes, nanoparticles, and viral protein particles, for example for delivery of antibodies, compounds, immunogens and nucleic acids described herein. In particular synthetic polymer vesicles, including polymersomes, can be used to administer antibodies.

Also provided in another aspect is a cell, optionally an isolated and/or recombinant cell, expressing an antibody described herein or comprising a vector herein disclosed.

The recombinant cell can be generated using any cell suitable for producing a polypeptide, for example suitable for producing an antibody and/or binding fragment thereof. For example to introduce a nucleic acid (e.g. a vector) into a cell, the cell may be transfected, transformed or infected, depending upon the vector employed.

Suitable host cells include a wide variety of prokaryotic and eukaryotic host cells. For example, the proteins described herein may be expressed in bacterial cells such as E. coli, insect cells (using baculovirus), yeast cells or mammalian cells.

In an embodiment, the cell is a eukaryotic cell selected from a yeast, plant, worm, insect, avian, fish, reptile and mammalian cell.

In another embodiment, the mammalian cell is a myeloma cell, a spleen cell, or a hybridoma cell.

In an embodiment, the cell is a neural cell.

Yeast and fungi host cells suitable for expressing an antibody or peptide include, but are not limited to Saccharomyces cerevisiae, Schizosaccharomyces pombe, the genera Pichia or Kluyveromyces and various species of the genus Aspergillus. Examples of vectors for expression in yeast S. cerivisiae include pYepSec1, pMFa, pJRY88, and pYES2 (Invitrogen Corporation, San Diego, Calif.). Protocols for the transformation of yeast and fungi are well known to those of ordinary skill in the art.

Mammalian cells that may be suitable include, among others: COS (e.g., ATCC No. CRL 1650 or 1651), BHK (e.g. ATCC No. CRL 6281), CHO (ATCC No. CCL 61), HeLa (e.g., ATCC No. CCL 2), 293 (ATCC No. 1573) and NS-1 cells. Suitable expression vectors for directing expression in mammalian cells generally include a promoter (e.g., derived from viral material such as polyoma, Adenovirus 2, cytomegalovirus and Simian Virus 40), as well as other transcriptional and translational control sequences. Examples of mammalian expression vectors include pCDM8 and pMT2PC.

In an embodiment, the cell is a fused cell such as a hybridoma cell, the hybridoma cell producing an antibody specific and/or selective for an epitope or epitope sequence described herein, including for example that selectively binds A-beta oligomers over A-beta monomers, selectively binds an epitope sequence presented in a cyclic compound relative to a linear compound or lacks or has negligible plaque binding.

A further aspect is a hybridoma cell line producing an antibody described herein.

### III. Compositions

A further aspect is a composition comprising a nucleic acid, vector or antibody described herein.

In an embodiment, the composition comprises a diluent.

Suitable diluents for nucleic acids include but are not limited to water, saline solutions and ethanol.

Suitable diluents for polypeptides, including antibodies or fragments thereof and/or cells include but are not limited to saline solutions, pH buffered solutions and glycerol solutions or other solutions suitable for freezing polypeptides and/or cells.

In an embodiment, the composition is a pharmaceutical composition comprising any of the antibodies, nucleic acids or vectors disclosed herein, and optionally comprising a pharmaceutically acceptable carrier.

The compositions described herein can be prepared by per se known methods for the preparation of pharmaceutically acceptable compositions that can be administered to subjects, optionally as a vaccine, such that an effective quantity of the active substance is combined in a mixture with a pharmaceutically acceptable vehicle.

Pharmaceutical compositions include, without limitation, lyophilized powders or aqueous or non-aqueous sterile injectable solutions or suspensions, which may further contain antioxidants, buffers, bacteriostats and solutes that render the compositions substantially compatible with the tissues or the blood of an intended recipient. Other components that may be present in such compositions include water, surfactants (such as Tween), alcohols, polyols, glycerin and vegetable oils, for example. Extemporaneous injection solutions and suspensions may be prepared from sterile powders, granules, tablets, or concentrated solutions or suspensions. The composition may be supplied, for example but not by way of limitation, as a lyophilized powder which is reconstituted with sterile water or saline prior to administration to the patient.

Pharmaceutical compositions may comprise a pharmaceutically acceptable carrier. Suitable pharmaceutically acceptable carriers include essentially chemically inert and nontoxic compositions that do not interfere with the effectiveness of the biological activity of the pharmaceutical composition. Examples of suitable pharmaceutical carriers include, but are not limited to, water, saline solutions, glycerol solutions, ethanol, N-(1(2,3-dioleyloxy)propyl)N,N,N-trimethylammonium chloride (DOTMA), diolesylphosphotidyl-ethanolamine (DOPE), and liposomes. Such compositions should contain a therapeutically effective amount of the compound, together with a suitable amount of carrier so as to provide the form for direct administration to the patient.

The composition may be in the form of a pharmaceutically acceptable salt which includes, without limitation, those formed with free amino groups such as those derived from hydrochloric, phosphoric, acetic, oxalic, tartaric acids, etc., and those formed with free carboxyl groups such as those derived from sodium, potassium, ammonium, calcium, ferric hydroxides, isopropylamine, triethylamine, 2-ethylarnino ethanol,

In an embodiment, the composition comprises an antibody described herein. In another embodiment, the composition comprises an antibody described herein and a diluent. In an embodiment, the composition is a sterile composition.

A further aspect includes an antibody complex comprising an antibody described herein and A-beta, optionally A-beta oligomer. The complex may be in solution or comprised in a tissue, optionally in vitro.

### IV. Kits

A further aspect relates to a kit comprising i) an antibody and/or binding fragment thereof disclosed herein, ii) a nucleic acid of said antibody or a part thereof, iii) composition comprising an antibody, nucleic acid or cell described herein or iv) a recombinant cell described herein, comprised in a vial such as a sterile vial or other housing and optionally a reference agent and/or instructions for use thereof.

In an embodiment, the kit further comprises one or more of a collection vial, standard buffer and detection reagent.

In another embodiment, the kit is for diagnosing or monitoring Alzheimer's disease or a condition involving oligomeric Abeta.

### V. Methods

A further aspect provides a method of detecting whether a biological sample comprises oligomeric A-beta the method comprising contacting the biological sample with an antibody described herein and/or detecting the presence of any antibody complex.

In an embodiment, the method comprises:

a. contacting the biologic sample with an antibody described herein that is specific and/or selective for A-beta oligomer herein under conditions permissive to produce an antibody: A-beta oligomer complex; and

b. detecting the presence of any complex;

wherein the presence of detectable complex is indicative that the sample may contain A-beta oligomer.

In an embodiment, the level of complex formed is compared to a test antibody such as a suitable Ig control or irrelevant antibody.

In an embodiment, the detection is quantitated and the amount of complex produced is measured. The measurement can for example be relative to a standard.

In an embodiment, the measured amount is compared to a control.

In another embodiment, the method comprises:

(a) contacting a test sample of said subject with an antibody described herein, under conditions permissive to produce an antibody-antigen complex;

(b) measuring the amount of the antibody-antigen complex in the test sample; and

(c) comparing the amount of antibody-antigen complex in the test sample to a control;

wherein detecting antibody-antigen complex in the test sample as compared to the control indicates that the sample comprises A-beta.

The control can be a sample control (e.g. from a subject without AD, or from a subject with a particular form of AD, mild, moderate or advanced), or be a previous sample from the same subject for monitoring changes in A-beta oligomer levels in the subject. Alternatively the control can be a value derived from a plurality of patients with or without AD.

In an embodiment, the antibody is an antibody having the CDR sequences described herein. In an embodiment, the antibody is a humanized antibody. In an embodiment, the antibody is a chimeric antibody.

In an embodiment, the sample is a biological sample. In an embodiment, the sample comprises brain tissue or an extract thereof and/or CSF. In an embodiment, the sample comprises whole blood, plasma or serum. In an embodiment, the sample is obtained from a human subject. In an embodiment, the subject is suspected of, at a risk of or has AD.

A number of methods can be used to detect an A-beta: antibody complex and thereby determine A-beta oligomers is present in a sample using the antibodies described herein, including immunoassays such as flow cytometry, Western blots, ELISA, SPR and immunoprecipitation followed by SDS-PAGE immunocytochemistry.

As described in the Examples surface plasmon resonance technology can be used to assess conformation specific binding. If the antibody is labeled or a detectably labeled secondary antibody specific for the complex antibody is used, the label can be detected. Commonly used reagents include fluorescent emitting and HRP labeled antibodies. In quantitative methods, the amount of signal produced can be measured by comparison to a standard or control. The measurement can also be relative.

A further aspect includes a method of measuring a level of or imaging A-beta in a subject or tissue, optionally where the A-beta to be measured or imaged is oligomeric A-beta. In an embodiment, the method comprises administering to a subject at risk or suspected of having or having AD, an antibody described herein conjugated to a detectable label; and detecting the label, optionally quantitatively detecting the label. The label in an embodiment is a positron emitting radionuclide which can for example be used in PET imaging.

The methods may also be combined with other tests for AD or cognitive impairment. For example, synaptic protein levels, such as SNAP-25 or synaptic vesicle glycoprotein 2a (SVG2a) (Sci Transl Med. 2016 Jul. 20; 8(348):348ra96. doi: 10.1126/scitranslmed.aaf6667) in CSF can be measured. For example, fluorodeoxyglucose PET (FDG-PET) is used as an indirect measure of synaptic metabolism.

Detecting A-beta levels using an antibody described herein can be used alone or in combination with other methods to monitor response to treatment.

Antibodies raised against cyclo(CGHHQKG) (SEQ ID NO: 2) have been shown to specifically and/or selectively bind A-beta oligomers and inhibit A-beta aggregation and A-beta oligomer propagation, as shown for example in WO2017/079833 or WO 2018/014126. Oligomeric A-beta species are believed to be the toxic propagating species in AD. Accordingly, also provided are methods of inhibiting A-beta oligomer propagation, the method comprising contacting a cell or tissue expressing A-beta with or administering to a subject in need thereof an effective amount of an A-beta oligomer specific or selective antibody described herein to inhibit A-beta aggregation and/or oligomer propagation.

The antibodies may also be useful for treating AD and/or other A-beta amyloid related diseases. For example, variants of Lewy body dementia and in inclusion body myositis (a muscle disease) exhibit similar plaques as AD and A-beta can also form aggregates implicated in cerebral amyloid angiopathy. As mentioned, the antibodies including humanized antibodies described herein bind oligomeric A-beta which is believed to be a toxigenic species of A-beta in AD and inhibit formation of toxigenic A-beta oligomers in vitro.

Accordingly, a further aspect is a method of treating AD and/or other A-beta amyloid related diseases, the method comprising administering to a subject in need thereof an effective amount of an antibody described herein, or a pharmaceutical composition comprising said antibody, to a subject in need thereof. In other embodiments, nucleic acids encoding the antibodies described herein can also be administered to the subject, optionally using vectors suitable for delivering nucleic acids in a subject.

In an embodiment, a biological sample from the subject to be treated is assessed for the presence or levels of A-beta using an antibody described herein. In an embodiment, a subject with detectable A-beta levels (e.g. A-beta antibody complexes measured in vitro or measured by imaging) is treated with the antibody.

The antibody, peptides and nucleic acids can for example be comprised in a pharmaceutical composition as described herein, and formulated for example in vesicles for improving delivery.

One or more antibodies can be administered in combination. In addition, the antibodies disclosed herein can be administered with one or more other treatments such as a beta-secretase inhibitor or a cholinesterase inhibitor.

Also provided are uses of the compositions, antibodies, isolated peptides, and nucleic acids for treating AD or A-beta amyloid related diseases.

The compositions, antibodies, isolated peptides and nucleic acids, vectors etc. described herein can be administered for example, by parenteral, intravenous, subcutaneous, intramuscular, intracranial, intraventricular, intrathecal, intraorbital, ophthalmic, intraspinal, intracisternal, intraperitoneal, intranasal, aerosol or oral administration.

In certain embodiments, the pharmaceutical composition is administered systemically.

In other embodiments, the pharmaceutical composition is administered directly to the brain or other portion of the CNS. For example, such methods include the use of an implantable catheter and a pump, which would serve to discharge a pre-determined dose through the catheter to the infusion site. A person skilled in the art would further recognize that the catheter may be implanted by surgical techniques that permit visualization of the catheter so as to position the catheter adjacent to the desired site of administration or infusion in the brain. Such techniques are described in Elsberry et al. U.S. Pat. No. 5,814,014 “Techniques of Treating Neurodegenerative Disorders by Brain Infusion”, which is herein incorporated by reference. Also contemplated are methods such as those described in US patent application 20060129126 (Kaplitt and During “Infusion device and method for infusing material into the brain of a patient”. Devices for delivering drugs to the brain and other parts of the CNS are commercially available (eg. SynchroMed® EL Infusion System; Medtronic, Minneapolis, Minn.).

In another embodiment, the pharmaceutical composition is administered to the brain using methods such as modifying the compounds to be administered to allow receptor-mediated transport across the blood brain barrier.

Other embodiments contemplate the co-administration of the compositions, antibodies, isolated peptides and nucleic acids described herein with biologically active molecules known to facilitate the transport across the blood brain barrier.

Also contemplated in certain embodiments, are methods for administering the compositions, antibodies, isolated peptides, and nucleic acids described herein across the blood brain barrier such as those directed at transiently increasing the permeability of the blood brain barrier as described in U.S. Pat. No. 7,012,061 “Method for increasing the permeability of the blood brain barrier”, herein incorporated by reference.

The above disclosure generally describes the present application. A more complete understanding can be obtained by reference to the following specific examples. These examples are described solely for the purpose of illustration and are not intended to limit the scope of the application. Changes in form and substitution of equivalents are contemplated as circumstances might suggest or render expedient. Although specific terms have been employed herein, such terms are intended in a descriptive sense and not for purposes of limitation.

The following non-limiting examples are illustrative of the present disclosure:

### EXAMPLES

### Example 1

### Affinity Maturation

### Methods and Materials

Affinity maturation was performed with the goal of increasing the affinity for the cyclic peptide target while minimizing/avoiding reactivity with the linear (non-conformational) form of the same peptide. Using phage display, selections were performed and single chain variable fragment (scFv) were identified that showed an improved binding profile compared to the parental scFv.

Briefly, scFv was derived from the PMN310 humanized antibody variant VH2/Vk5 consisting of the parental antibody VH and Vk linked by a 15 amino acid (G₄S)₃ linker. The sequences of the variable heavy chain and variable light chain of the parental antibody are provided in Table 1. The CDR sequences, defined based on IMGT numbering scheme, are bolded and underlined in Table 1. In Table 2, the CDR sequences of the parental antibody as defined using IMGT are provided, along with their corresponding CDR sequences as defined using Kabat. Three phage libraries were constructed (2 targeting VH CDR3 and one targeting VL CDR3) by mutagenesis and screened against cyclic peptide-BSA for positive selection and linear peptide-BSA and BSA as selection controls. Three successive rounds of selection were performed.

TABLE 1
Sequences of variable heavy chain and variable light
chain of parental antibody used in the Examples described herein

| Parental | | Polypeptide |
|---|---|---|
| Antibody | cDNA Sequence | Sequence |
| VH2 | CAGGTCCAACTGGTGCAGTCTGGGGCTGAGGTGAAGAAGCCTGGG | QVQLVQSGAEVKKP |
| SEQ ID NO: 71, 3 | GCTTCAGTGAAGATGTCCTGCAAGGCTTCT [symbol] | GASVKMSCKAS [symbol] |
| | [symbol] ATAAACTGGGTGAAGCAGAGGCCTGGACAAGGCCTT | [symbol] INWVKQRPG |
| | GAGTGGATTGGAGAT [symbol] [symbol] TAC | QGLEWIGD [symbol] |
| | AATGCTAAGTTCAAGAGCAGAGCCACACTGACTCTGGACACATCC | [symbol] YNAKFKSRATL |
| | ATAAGCACAGCCTACATGGAGCTCAGCAGCCTGAGATCTGAGGAC | TLDTSISTAYMELS |
| | ACGGCGGTCTATTACTGT [symbol] [symbol] | SLRSEDTAVYYC [symbol] |
| | [symbol] TGGGGCCAAGGCACCACGGTCACCGTCTCCTCA | [symbol] WGQ |
| | | GTTVTVSS |
| VK5 | GATGTTCTGATGACCCAATCTCCACTCTCCCTGCCTGTCACCCTT | DVLMTQSPLSLPVT |
| SEQ ID NO: 72, 4 | GGACAGCCGGCCTCCATCTCTTGCAGATCTAGT [symbol] | LGQPASISCRSS [symbol] |
| | [symbol] [symbol] TTAGAATGGTACCAGCAGAGGCCA | [symbol] LEWYQ |
| | GGCCAGTCTCCAAGGCTGCTGATCTAC [symbol] AACCGATTT | QRPGQSPRLLIY [symbol] |
| | TCTGGGGTCCCAGACAGGTTCAGTGGCAGTGGATCAGGGACAGAT | [symbol] NRFSGVPDRFSGS |
| | TTCACACTCAAGATCAGCAGAGTGGAGGCTGAGGATGTTGGAGTT | GSGTDFTLKISRVE |
| | TATTACTGC [symbol] [symbol] TTTGGCCAA | AEDVGVYYC [symbol] |
| | GGGACCAAGCTGGAGATCAAA | [symbol] FGQGTKLEIK |

TABLE 2
CDR sequences of parental antibody

| Chain | CDR | IMGT Sequence | SEQ ID NO. | Kabat Sequence | SEQ ID NO. |
|---|---|---|---|---|---|
| Heavy | CDR-H1 | GYSFTSYW | 5 | SYWIN | 84 |
| | CDR-H2 | VHPGRGVST | 6 | DVHPGRGVSTYNAKFKS | 85 |
| | CDR-H3 | SRSHGNTYWFFDV | 7 | GALSPYWFFDV | 86 |
| Light | CDR-L1 | QSIVHSNGNTY | 8 | RSSQSIVHSNGNTYLE | 87 |
| | CDR-L2 | KVS | 9 | KVSNRFS | 88 |
| | CDR-L3 | FQGSHVPFT | 10 | FQGSHVPFT | 89 |

### Preliminary Binding Assays

More than 5000 clones were screened in the primary binding ELISA after completing the selections. Selections were successful for two of the three libraries made both targeting VH CDR3. All output sequences in the VL CDR3 library were parental indicating that improvements were not achieved by targeting the VL CDR3. The 440 clones showing the greatest signal in the primary binding ELISA were selected and grown in 96 well plates for further screening against cyclo-BSA peptide, linear-BSA peptide and BSA. Parental scFv and an irrelevant scFv (negative control) were used as a controls in all the plates. Peri preps containing expressed scFv were diluted 1 in 15 for the ELISA.

ELISAs were carried out as follows: 1) Cyclo-BSA, linear-BSA or BSA (non-specific control) was coated on a 96W plate overnight at 0.5 μg/ml. 2) Non-specific binding was blocked with 5% milk Dulbecco's PBS for 1 hr at RT. 3) 100 μl of a dilution 1 in 15 of the peri prep was transferred to all three blocked antigen plates and incubated for 1.5 hr at room temperature. 4) scFv binding was detected with anti-flag HRP antibody (1:20000). 5) The plate was developed with TMB and stopped with 3M HCl.

### Sequencing and IgG Preparation

The top twenty scFV hits with the highest binding to cyclic peptide-BSA and reduced binding to linear peptide-BSA were sequenced and converted to an IgG format for further characterization.

Briefly, the humanized variable region genes were cloned into vectors encoding a human IgG4 (S241P hinge variant) heavy chain constant domain and a human kappa light chain constant domain. Plasmids encoding the 20-affinity matured VH variants (and parental V_{H}) were transiently transfected together with the plasmid encoding the parental Vk into HEK EBNA adherent cells. Supernatants were harvested 7 days later for testing. The S241P hinge variant comprises an altered disulfide bond arrangement of an IgG4 molecule by mutation of the Cys at the N terminus of the heavy chain constant domain 1 (CH1) (Kabat position 127) to a Ser and introduction of a Cys at a variety of positions (positions 227-230) at the C terminus of CH1. An inter-LC-CH1 disulfide bond is formed (Peters S J et al).

### Single Cycle Kinetic (SCK) Analysis

SCK was first performed on cell supernatants by SPR to assess binding to cyclo (CGHHQKG) (SEQ ID NO: 2)-BSA and linear peptide-BSA.

Briefly, antibodies in the supernatant were captured on a Protein A chip (immobilization level of ˜112 RU) and SCK analysis was performed with both cyclic peptide-BSA and linear peptide-BSA injected over the surface at a flow rate of 30 μI/min. A four-point, three-fold dilution range from 0.025 nM to 0.675 nM peptide-BSA without regeneration between each concentration was used. For analysis, the assumption was made that only one cyclic or linear peptide molecule is capable of binding to the antibody i.e. one epitope per molecule.

### Results

### ELISA Antibody Screening

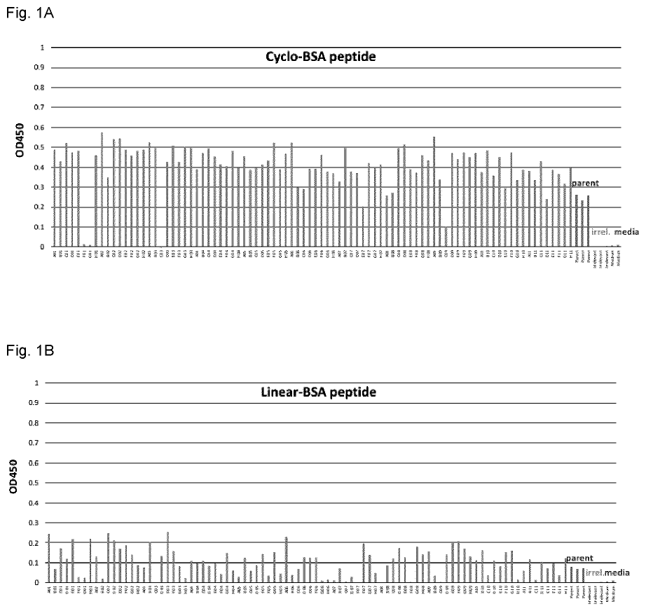

ELISA assays were used to confirm that the clones preferentially bound the cyclopeptide relative to the linear peptide. Representative results are shown in FIG. 1A and FIG. 1B. None of the 440 selected clones bound BSA (not shown).

### Single Cycle Kinetic (SCK) Analysis

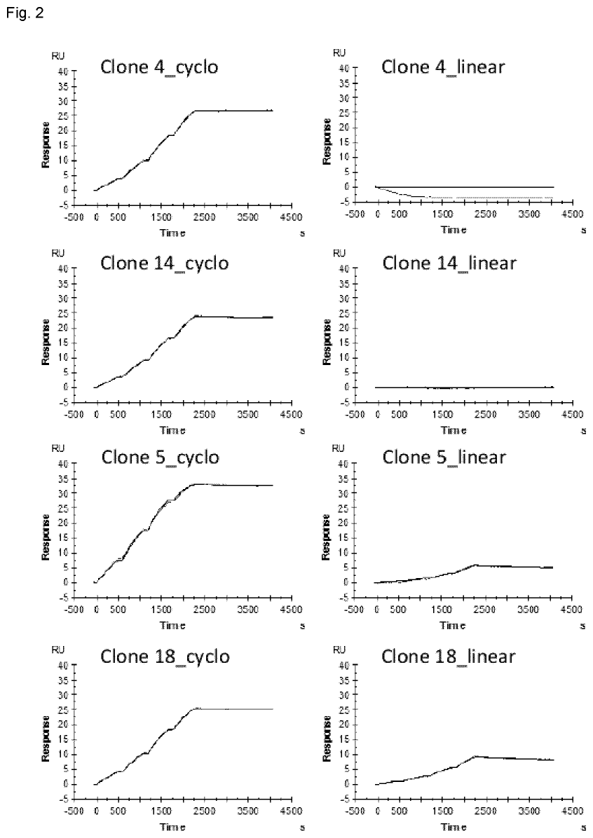

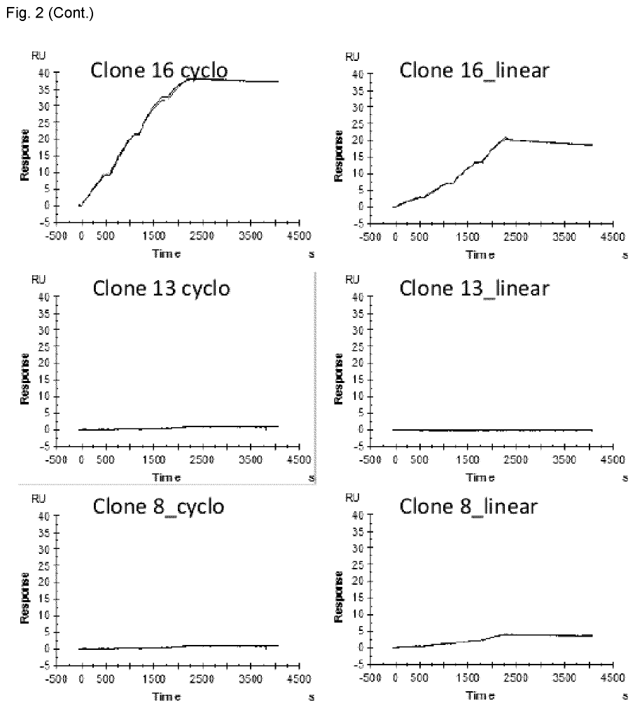

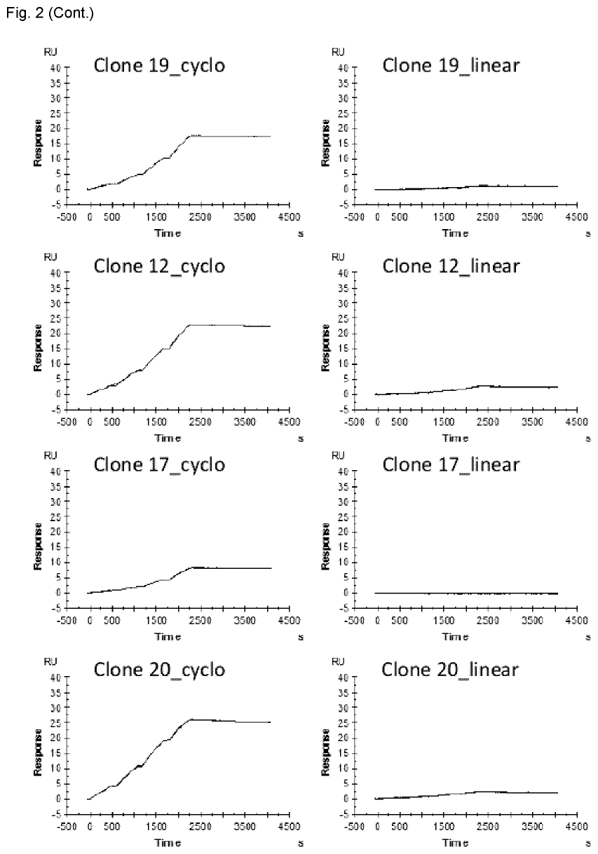

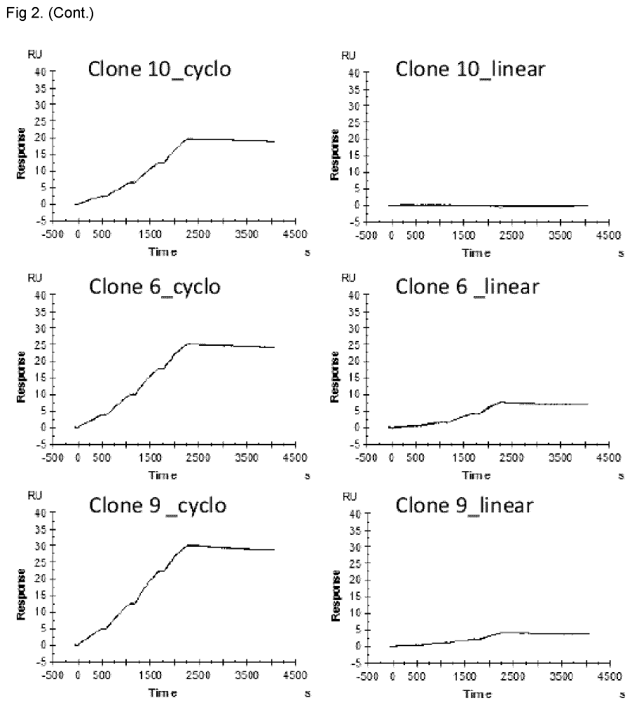

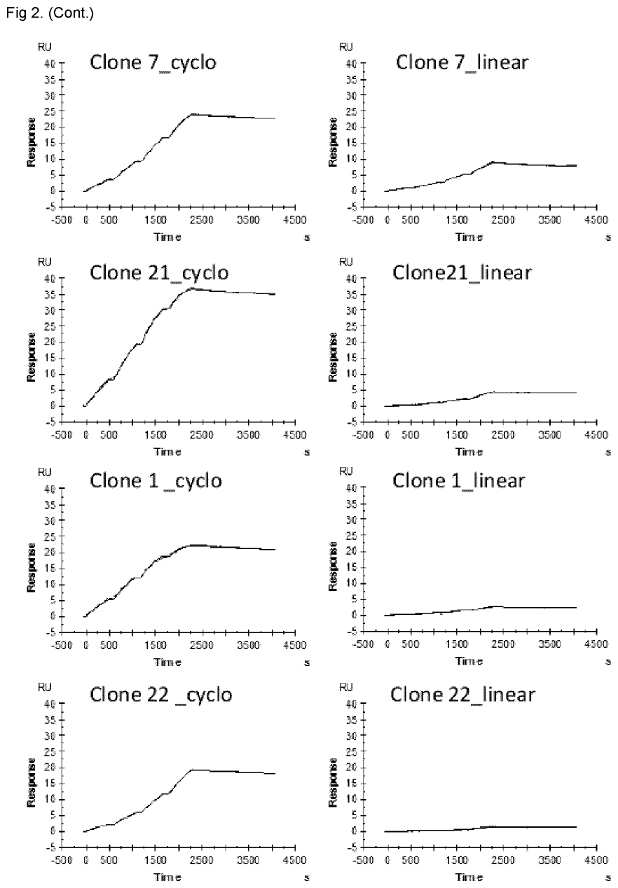

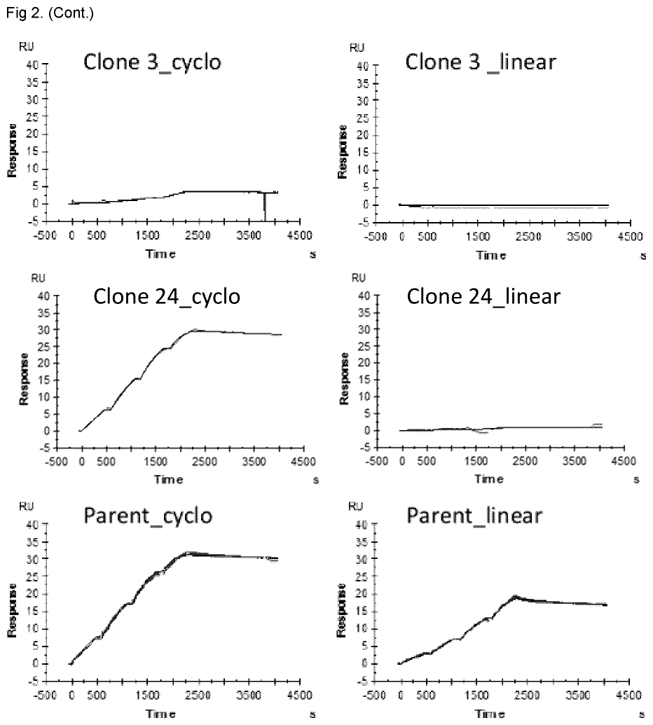

Sensorgrams for the parental antibody and clones formatted as IgG are shown in FIG. 2. Most of the IgGs screened bound with similar, or better, affinities to the target cyclo-peptide cyclo (CGHHQKG) (SEQ ID NO:2)-BSA. The affinity to the linear peptide differs among all the clones screened. Some clones did not demonstrate appreciable binding to the linear peptide, indicating an increased selectivity for cyclo- versus linear-peptide.

Sequences of the heavy chain variable domains and CDR3s for each clone are shown in Tables 3 Table 4, respectively. In Table 3, the complementarity determining region 3 (CDR3) according to IMTG/LIGM-DB is underlined in each polypeptide sequence. In Table 4, the CDR-H3 sequences of selected clones as defined using IMGT are provided, along with their corresponding CDR sequences as defined using Kabat.

TABLE 3
Protein sequences of the heavy chain variable region

| Antibody | SEQ ID NO: | Polypeptide sequence |
|---|---|---|
| Clone 1 | 11 | QVQLVQSGAEVKKPGASVKMSCKASGYSFTSYWINWVKQRPGQGLEWIGDVH |
| | | PGRGVSTYNAKFKSRATLTLDTSISTAYMELSSLRSEDTAVYYC [symbol] |
| | | [symbol] WGQGTTVTVSS |
| Clone 3 | 12 | QVQLVQSGAEVKKPGASVKMSCKASGYSFTSYWINWVKQRPGQGLEWIGDVH |
| | | PGRGVSTYNAKFKSRATLTLDTSISTAYMELSSLRSEDTAVYYC |
| | | [symbol] WGQGTTVTVSS |
| Clone 4 | 13 | QVQLVQSGAEVKKPGASVKMSCKASGYSFTSYWINWVKQRPGQGLEWIGDVH |
| | | PGRGVSTYNAKFKSRATLTLDTSISTAYMELSSLRSEDTAVYYC |
| | | [symbol] WGQGTTVTVSS |
| Clone 5 | 14 | QVQLVQSGAEVKKPGASVKMSCKASGYSFTSYWINWVKQRPGQGLEWIGDVH |
| | | PGRGVSTYNAKFKSRATLTLDTSISTAYMELSSLRSEDTAVYYC |
| | | [symbol] WGQGTTVTVSS |
| Clone 6 | 15 | QVQLVQSGAEVKKPGASVKMSCKASGYSFTSYWINWVKQRPGQGLEWIGDVH |
| | | PGRGVSTYNAKFKSRATLTLDTSISTAYMELSSLRSEDTAVYYC |
| | | [symbol] WGQGTTVTVSS |
| Clone 7 | 16 | QVQLVQSGAEVKKPGASVKMSCKASGYSFTSYWINWVKQRPGQGLEWIGDVH |
| | | PGRGVSTYNAKFKSRATLTLDTSISTAYMELSSLRSEDTAVYYC |
| | | [symbol] WGQGTTVTVSS |
| Clone 8 | 17 | QVQLVQSGAEVKKPGASVKMSCKASGYSFTSYWINWVKQRPGQGLEWIGDVH |
| | | PGRGVSTYNAKFKSRATLTLDTSISTAYMELSSLRSEDTAVYYC |
| | | [symbol] WGQGTTVTVSS |
| Clone 9 | 18 | QVQLVQSGAEVKKPGASVKMSCKASGYSFTSYWINWVKQRPGQGLEWIGDVH |
| | | PGRGVSTYNAKFKSRATLTLDTSISTAYMELSSLRSEDTAVYYC |
| | | [symbol] WGQGTTVTVSS |
| Clone 10 | 19 | QVQLVQSGAEVKKPGASVKMSCKASGYSFTSYWINWVKQRPGQGLEWIGDVH |
| | | PGRGVSTYNAKFKSRATLTLDTSISTAYMELSSLRSEDTAVYYC |
| | | [symbol] WGQGTTVTVSS |
| Clone 12 | 20 | QVQLVQSGAEVKKPGASVKMSCKASGYSFTSYWINWVKQRPGQGLEWIGDVH |
| | | PGRGVSTYNAKFKSRATLTLDTSISTAYMELSSLRSEDTAVYYC |
| | | [symbol] WGQGTTVTVSS |
| Clone 13 | 21 | QVQLVQSGAEVKKPGASVKMSCKASGYSFTSYWINWVKQRPGQGLEWIGDVH |
| | | PGRGVSTYNAKFKSRATLTLDTSISTAYMELSSLRSEDTAVYYC |
| | | [symbol] WGQGTTVTVSS |
| Clone 14 | 22 | QVQLVQSGAEVKKPGASVKMSCKASGYSFTSYWINWVKQRPGQGLEWIGDVH |
| | | PGRGVSTYNAKFKSRATLTLDTSISTAYMELSSLRSEDTAVYYC |
| | | [symbol] WGQGTTVTVSS |
| Clone 16 | 23 | QVQLVQSGAEVKKPGASVKMSCKASGYSFTSYWINWVKQRPGQGLEWIGDVH |
| | | PGRGVSTYNAKFKSRATLTLDTSISTAYMELSSLRSEDTAVYYC |
| | | [symbol] WGQGTTVTVSS |
| Clone 17 | 24 | QVQLVQSGAEVKKPGASVKMSCKASGYSFTSYWINWVKQRPGQGLEWIGDVH |
| | | PGRGVSTYNAKFKSRATLTLDTSISTAYMELSSLRSEDTAVYYC |
| | | [symbol] WGQGTTVTVSS |
| Clone 18 | 25 | QVQLVQSGAEVKKPGASVKMSCKASGYSFTSYWINWVKQRPGQGLEWIGDVH |
| | | PGRGVSTYNAKFKSRATLTLDTSISTAYMELSSLRSEDTAVYYC |
| | | [symbol] WGQGTTVTVSS |
| Clone 19 | 26 | QVQLVQSGAEVKKPGASVKMSCKASGYSFTSYWINWVKQRPGQGLEWIGDVH |
| | | PGRGVSTYNAKFKSRATLTLDTSISTAYMELSSLRSEDTAVYYC |
| | | [symbol] WGQGTTVTVSS |
| Clone 20 | 27 | QVQLVQSGAEVKKPGASVKMSCKASGYSFTSYWINWVKQRPGQGLEWIGDVH |
| | | PGRGVSTYNAKFKSRATLTLDTSISTAYMELSSLRSEDTAVYYC |
| | | [symbol] WGQGTTVTVSS |
| Clone 21 | 28 | QVQLVQSGAEVKKPGASVKMSCKASGYSFTSYWINWVKQRPGQGLEWIGDVH |
| | | PGRGVSTYNAKFKSRATLTLDTSTSTAYMELSSLRSEDTAVYYC |
| | | [symbol] WGQGTTVTVSS |
| Clone 22 | 29 | QVQLVQSGAEVKKPGASVKMSCKASGYSFTSYWINWVKQRPGQGLEWIGDVH |
| | | PGRGVSTYNAKFKSRATLTLDTSISTAYMELSSLRSEDTAVYYC |
| | | [symbol] WGQGTTVTVSS |
| Clone 24 | 30 | QVQLVQSGAEVKKPGASVKMSCKASGYSFTSYWINWVKQRPGQGLEWIGDVH |
| | | PGRGVSTYNAKFKSRATLTLDTSISTAYMELSSLRSEDTAVYYC |
| | | [symbol] WGQGTTVTVSS |

TABLE 4
CDR-H3 sequences of selected clones

| | | SEQ | | SEQ |
|---|---|---|---|---|
| Clone | IMGT Sequence | ID NO. | Kabat Sequence. | ID NO. |
| Parental | SRSHGNTYWFFDV | 7 | GALSPYWFFDV | 86 |
| 1 | ARGALSPYWFFDV | 31 | GALSPYWFFDV | 90 |
| 3 | ARSKTPVYWFFDV | 32 | SKTPVYWFFDV | 91 |
| 4 | ARVSPTGYWFFDV | 33 | VSPTGYWFFDV | 92 |
| 5 | ARGALSPYWFFDV | 34 | GALSPYWFFDV | 93 |
| 6 | SRSSPFNYWFFDV | 35 | SSPFNYWFFDV | 94 |
| 7 | ARSPSSTYWFFDV | 36 | SPSSTYWFFDV | 95 |
| 8 | ARGAMSPYWFFDV | 37 | GAMSPYWFFDV | 96 |
| 9 | AKSPQQSYWFFDV | 38 | SPQQSYWFFDV | 97 |
| 10 | ARSTTTGYWFFDV | 39 | STTTGYWFFDV | 98 |
| 12 | ARSSAQDYWFFDV | 40 | SSAQDYWFFDV | 99 |
| 13 | ARSVGDTYWFYDV | 41 | SVGDTYWFYDV | 100 |
| 14 | AKSDASGYWFFDV | 42 | SDASGYWFFDV | 101 |
| 16 | SRSHGNTPWFLDD | 43 | SHGNTPWFLDD | 102 |
| 17 | ARSSGGRYWFFDV | 44 | SSGGRYWFFDV | 103 |
| 18 | SRSHGNSPAFFDV | 45 | SHGNSPAFFDV | 104 |
| 19 | ARSMSQNYWFFDV | 46 | SMSQNYWFFDV | 105 |
| 20 | ARSSVHGYWFFDV | 47 | SSVHGYWFFDV | 106 |
| 21 | SRSHGNRPWFYDF | 48 | SHGNRPWFYDF | 107 |
| 22 | ARSSHKGYWFFDV | 49 | SSHKGYWFFDV | 108 |
| 24 | ARVSPMSYWFFDV | 50 | VSPMSYWFFDV | 109 |

TABLE 5
DNA sequences of the heavy chain variable domain are provided

| | SEQ | |
|---|---|---|
| Antibody | ID NO: | DNA sequence |
| Clone 1 | 51 | C A G G T C C A A C T G G T G C A G T C T G G G G C T G A G G T G A A G |
| | | A A G C C T G G G G C T T C A G T G A A G A T G T C C T G C A A G G C T |
| | | T C T G G C T A C A G C T T C A C C A G C T A C T G G AT A A A C T G G |
| | | G T G A A G C A G A G G C C T G G A C A A G G C C T T G A G T G G A T T |
| | | G G A G A T G T G C A T C C T G G T A G A G G C G T G T C C A C A T A C |
| | | A A T G C T A A G T T C A A G A G C A GA G C C A C A C T G A C T C T G |
| | | G A C A C A T C C A T A A G C A C A G C C T A C A T G G A G C T C A G C |
| | | A G C C T G A G A T C T G A G G A C A C G G C G G T C T A T T A C T G T |
| | | G C C A G G G G C G C CC T C A G T C C C T A C T G G T T T T T T G A C |
| | | G T C T G G G G C C A A G G C A C C A C G G T C A C C G T C T C C T C A |
| Clone 3 | 52 | C A G G T C C A A C T G G T G C A G T C T G G G G C T G A G G T G A A G |
| | | A A G C C T G G G G C T T C A G T G A A G A T G T C C T G C A A G G C T |
| | | T C T G G C T A C A G C T T C A C C A G C T A C T G G AT A A A C T G G |
| | | G T G A A G C A G A G G C C T G G A C A A G G C C T T G A G T G G A T T |
| | | G G A G A T G T G C A T C C T G G T A G A G G C G T G T C C A C A T A C |
| | | A A T G C T A A G T T C A A G A G C A GA G C C A C A C T G A C T C T G |
| | | G A C A C A T C C A T A A G C A C A G C C T A C A T G G A G C T C A G C |
| | | A G C C T G A G A T C T G A G G A C A C G G C G G T C T A T T A C T G T |
| | | G C C A G G T C C A A GA C G C C G G T C T A C T G G T T T T T T G A C |
| | | G T C T G G G G C C A A G G C A C C A C G G T C A C C G T C T C C T C A |
| Clone 4 | 53 | C A G G T C C A A C T G G T G C A G T C T G G G G C T G A G G T G A A G |
| | | A A G C C T G G G G C T T C A G T G A A G A T G T C C T G C A A G G C T |
| | | T C T G G C T A C A G C T T C A C C A G C T A C T G G AT A A A C T G G |
| | | G T G A A G C A G A G G C C T G G A C A A G G C C T T G A G T G G A T T |
| | | G G A G A T G T G C A T C C T G G T A G A G G C G T G T C C A C A T A C |
| | | A A T G C T A A G T T C A A G A G C A GA G C C A C A C T G A C T C T G |
| | | G A C A C A T C C A T A A G C A C A G C C T A C A T G G A G C T C A G C |
| | | A G C C T G A G A T C T G A G G A C A C G G C G G T C T A T T A C T G T |
| | | G C C A G G G T C T C GC C G A C G G G C T A C T G G T T T T T T G A C |
| | | G T C T G G G G C C A A G G C A C C A C G G T C A C C G T C T C C T C A |
| Clone 5 | 54 | C A G G T C C A A C T G G T G C A G T C T G G G G C T G A G G T G A A G |
| | | A A G C C T G G G G C T T C A G T G A A G A T G T C C T G C A A G G C T |
| | | T C T G G C T A C A G C T T C A C C A G C T A C T G G AT A A A C T G G |
| | | G T G A A G C A G A G G C C T G G A C A A G G C C T T G A G T G G A T T |
| | | G G A G A T G T G C A T C C T G G T A G A G G C G T G T C C A C A T A C |
| | | A A T G C T A A G T T C A A G A G C A GA G C C A C A C T G A C T C T G |
| | | G A C A C A T C C A T A A G C A C A G C C T A C A T G G A G C T C A G C |
| | | A G C C T G A G A T C T G A G G A C A C G G C G G T C T A T T A C T G T |
| | | G C C A G G G G C G C GT T G T C T C C C T A C T G G T T T T T T G A C |
| | | G T C T G G G G C C A A G G C A C C A C G G T C A C C G T C T C C T C A |
| Clone 6 | 55 | C A G G T C C A A C T G G T G C A G T C T G G G G C T G A G G T G A A G |
| | | A A G C C T G G G G C T T C A G T G A A G A T G T C C T G C A A G G C T |
| | | T C T G G C T A C A G C T T C A C C A G C T A C T G G AT A A A C T G G |
| | | G T G A A G C A G A G G C C T G G A C A A G G C C T T G A G T G G A T T |
| | | G G A G A T G T G C A T C C T G G T A G A G G C G T G T C C A C A T A C |
| | | A A T G C T A A G T T C A A G A G C A GA G C C A C A C T G A C T C T G |
| | | G A C A C A T C C A T A A G C A C A G C C T A C A T G G A G C T C A G C |
| | | A G C C T G A G A T C T G A G G A C A C G G C G G T C T A T T A C T G T |
| | | T C C A G G T C C A G CC C G T T T A A C T A C T G G T T T T T T G A C |
| | | G T C T G G G G C C A A G G C A C C A C G G T C A C C G T C T C C T C A |
| Clone 7 | 56 | C A G G T C C A A C T G G T G C A G T C T G G G G C T G A G G T G A A G |
| | | A A G C C T G G G G C T T C A G T G A A G A T G T C C T G C A A G G C T |
| | | T C T G G C T A C A G C T T C A C C A G C T A C T G G AT A A A C T G G |
| | | G T G A A G C A G A G G C C T G G A C A A G G C C T T G A G T G G A T T |
| | | G G A G A T G T G C A T C C T G G T A G A G G C G T G T C C A C A T A C |
| | | A A T G C T A A G T T C A A G A G C A GA G C C A C A C T G A C T C T G |
| | | G A C A C A T C C A T A A G C A C A G C C T A C A T G G A G C T C A G C |
| | | A G C C T G A G A T C T G A G G A C A C G G C G G T C T A T T A C T G T |
| | | G C C A G G T C C C C GT C C T C G A C C T A C T G G T T T T T T G A C |
| | | G T C T G G G G C C A A G G C A C C A C G G T C A C C G T C T C C T C A |
| Clone 8 | 57 | C A G G T C C A A C T G G T G C A G T C T G G G G C T G A G G T G A A G |
| | | A A G C C T G G G G C T T C A G T G A A G A T G T C C T G C A A G G C T |
| | | T C T G G C T A C A G C T T C A C C A G C T A C T G G A T A A A C T G G |
| | | G T G A A G C A G A G G C C T G G A C A A G G C C T T G A G T G G A T T |
| | | G G A G A T G T G C A T C C T G G T A G A G G C G T G T C C A C A T A C |
| | | A A T G C T A A G T T C A A G A G C A G A G C C A C A C T G A C T C T G |
| | | G A C A C A T C C A T A A G C A C A G C C T A C A T G G A G C T C A G C |
| | | A G C C T G A G A T C T G A G G A C A C G G C G G T C T A T T A C T G T |
| | | G C C A G G G G C G C C A T G T C T C C C T A C T G G T T T T T T G A C |
| | | G T C T G G G G C C A A G G C A C C A C G G T C A C C G T C T C C T C A |
| Clone 9 | 58 | C A G G T C C A A C T G G T G C A G T C T G G G G C T G A G G T G A A G |
| | | A A G C C T G G G G C T T C A G T G A A G A T G T C C T G C A A G G C T |
| | | T C T G G C T A C A G C T T C A C C A G C T A C T G G AT A A A C T G G |
| | | G T G A A G C A G A G G C C T G G A C A A G G C C T T G A G T G G A T T |
| | | G G A G A T G T G C A T C C T G G T A G A G G C G T G T C C A C A T A C |
| | | A A T G C T A A G T T C A A G A G C A GA G C C A C A C T G A C T C T G |
| | | G A C A C A T C C A T A A G C A C A G C C T A C A T G G A G C T C A G C |
| | | A G C C T G A G A T C T G A G G A C A C G G C G G T C T A T T A C T G T |
| | | G C C A A G T C C C C CC A G C A G T C C T A C T G G T T T T T T G A C |
| | | G T C T G G G G C C A A G G C A C C A C G G T C A C C G T C T C C T C A |
| Clone 10 | 59 | C A G G T C C A A C T G G T G C A G T C T G G G G C T G A G G T G A A G |
| | | A A G C C T G G G G C T T C A G T G A A G A T G T C C T G C A A G G C T |
| | | T C T G G C T A C A G C T T C A C C A G C T A C T G G AT A A A C T G G |
| | | G T G A A G C A G A G G C C T G G A C A A G G C C T T G A G T G G A T T |
| | | G G A G A T G T G C A T C C T G G T A G A G G C G T G T C C A C A T A C |
| | | A A T G C T A A G T T C A A G A G C A GA G C C A C A C T G A C T C T G |
| | | G A C A C A T C C A T A A G C A C A G C C T A C A T G G A G C T C A G C |
| | | A G C C T G A G A T C T G A G G A C A C G G C G G T C T A T T A C T G T |
| | | G C C A G G T C C A C GA C G A C G G G C T A C T G G T T T T T T G A C |
| | | G T C T G G G G C C A A G G C A C C A C G G T C A C C G T C T C C T C A |
| Clone 12 | 60 | C A G G T C C A A C T G G T G C A G T C T G G G G C T G A G G T G A A G |
| | | A A G C C T G G G G C T T C A G T G A A G A T G T C C T G C A A G G C T |
| | | T C T G G C T A C A G C T T C A C C A G C T A C T G G AT A A A C T G G |
| | | G T G A A G C A G A G G C C T G G A C A A G G C C T T G A G T G G A T T |
| | | G G A G A T G T G C A T C C T G G T A G A G G C G T G T C C A C A T A C |
| | | A A T G C T A A G T T C A A G A G C A GA G C C A C A C T G A C T C T G |
| | | G A C A C A T C C A T A A G C A C A G C C T A C A T G G A G C T C A G C |
| | | A G C C T G A G A T C T G A G G A C A C G G C G G T C T A T T A C T G T |
| | | G C C A G G T C C T C CG C G C A G G A C T A C T G G T T T T T T G A C |
| | | G T C T G G G G C C A A G G C A C C A C G G T C A C C G T C T C C T C A |
| Clone 13 | 61 | C A G G T C C A A C T G G T G C A G T C T G G G G C T G A G G T G A A G |
| | | A A G C C T G G G G C T T C A G T G A A G A T G T C C T G C A A G G C T |
| | | T C T G G C T A C A G C T T C A C C A G C T A C T G G A T A A A C T G G |
| | | G T G A A G C A G A G G C C T G G A C A A G G C C T T G A G T G G A T T |
| | | G G A G A T G T G C A T C C T G G T A G A G G C G T G T C C A C A T A C |
| | | A A T G C T A A G T T C A A G A G C A G A G C C A C A C T G A C T C T G |
| | | G A C A C A T C C A T A A G C A C A G C C T A C A T G G A G C T C A G C |
| | | A G C C T G A G A T C T G A G G A C A C G G C G G T C T A T T A C T G T |
| | | G C C A G G T C C G T G G G G G A T A C C T A C T G G T T T T a T G A C |
| | | G T C T G G G G C C A A G G C A C C A C G G T C A C C G T C T C C T C A |
| Clone 14 | 62 | C A G G T C C A A C T G G T G C A G T C T G G G G C T G A G G T G A A G |
| | | A A G C C T G G G G C T T C A G T G A A G A T G T C C T G C A A G G C T |
| | | T C T G G C T A C A G C T T C A C C A G C T A C T G G AT A A A C T G G |
| | | G T G A A G C A G A G G C C T G G A C A A G G C C T T G A G T G G A T T |
| | | G G A G A T G T G C A T C C T G G T A G A G G C G T G T C C A C A T A C |
| | | A A T G C T A A G T T C A A G A G C A GA G C C A C A C T G A C T C T G |
| | | G A C A C A T C C A T A A G C A C A G C C T A C A T G G A G C T C A G C |
| | | A G C C T G A G A T C T G A G G A C A C G G C G G T C T A T T A C T G T |
| | | G C C A A G T C C G A CG C C T C G G G C T A C T G G T T T T T T G A C |
| | | G T C T G G G G C C A A G G C A C C A C G G T C A C C G T C T C C T C A |
| Clone 16 | 63 | C A G G T C C A A C T G G T G C A G T C T G G G G C T G A G G T G A A G |
| | | A A G C C T G G G G C T T C A G T G A A G A T G T C C T G C A A G G C T |
| | | T C T G G C T A C A G C T T C A C C A G C T A C T G G AT A A A C T G G |
| | | G T G A A G C A G A G G C C T G G A C A A G G C C T T G A G T G G A T T |
| | | G G A G A T G T G C A T C C T G G T A G A G G C G T G T C C A C A T A C |
| | | A A T G C T A A G T T C A A G A G C A GA G C C A C A C T G A C T C T G |
| | | G A C A C A T C C A T A A G C A C A G C C T A C A T G G A G C T C A G C |
| | | A G C C T G A G A T C T G A G G A C A C G G C G G T C T A T T A C T G T |
| | | A G C A G A T C T C A TG G T A A C A C C C C T T G G T T T C T C G A C |
| | | G A C T G G G G C C A A G G C A C C A C G G T C A C C G T C T C C T C A |
| Clone 17 | 64 | C A G G T C C A A C T G G T G C A G T C T G G G G C T G A G G T G A A G |
| | | A A G C C T G G G G C T T C A G T G A A G A T G T C C T G C A A G G C T |
| | | T C T G G C T A C A G C T T C A C C A G C T A C T G G AT A A A C T G G |
| | | G T G A A G C A G A G G C C T G G A C A A G G C C T T G A G T G G A T T |
| | | G G A G A T G T G C A T C C T G G T A G A G G C G T G T C C A C A T A C |
| | | A A T G C T A A G T T C A A G A G C A GA G C C A C A C T G A C T C T G |
| | | G A C A C A T C C A T A A G C A C A G C C T A C A T G G A G C T C A G C |
| | | A G C C T G A G A T C T G A G G A C A C G G C G G T C T A T T A C T G T |
| | | G C C A G G T C C T C CG G G G G T C G C T A C T G G T T T T T T G A C |
| | | G T C T G G G G C C A A G G C A C C A C G G T C A C C G T C T C C T C A |
| Clone 18 | 65 | C A G G T C C A A C T G G T G C A G T C T G G G G C T G A G G T G A A G |
| | | A A G C C T G G G G C T T C A G T G A A G A T G T C C T G C A A G G C T |
| | | T C T G G C T A C A G C T T C A C C A G C T A C T G G AT A A A C T G G |
| | | G T G A A G C A G A G G C C T G G A C A A G G C C T T G A G T G G A T T |
| | | G G A G A T G T G C A T C C T G G T A G A G G C G T G T C C A C A T A C |
| | | A A T G C T A A G T T C A A G A G C A GA G C C A C A C T G A C T C T G |
| | | G A C A C A T C C A T A A G C A C A G C C T A C A T G G A G C T C A G C |
| | | A G C C T G A G A T C T G A G G A C A C G G C G G T C T A T T A C T G T |
| | | A G C A G A T C T C A TG G T A A C T C C C C G G C G T T T T T C G A C |
| | | G T C T G G G G C C A A G G C A C C A C G G T C A C C G T C T C C T C A |
| Clone 19 | 66 | C A G G T C C A A C T G G T G C A G T C T G G G G C T G A G G T G A A G |
| | | A A G C C T G G G G C T T C A G T G A A G A T G T C C T G C A A G G C T |
| | | T C T G G C T A C A G C T T C A C C A G C T A C T G G AT A A A C T G G |
| | | G T G A A G C A G A G G C C T G G A C A A G G C C T T G A G T G G A T T |
| | | G G A G A T G T G C A T C C T G G T A G A G G C G T G T C C A C A T A C |
| | | A A T G C T A A G T T C A A G A G C A GA G C C A C A C T G A C T C T G |
| | | G A C A C A T C C A T A A G C A C A G C C T A C A T G G A G C T C A G C |
| | | A G C C T G A G A T C T G A G G A C A C G G C G G T C T A T T A C T G T |
| | | G C C A G G T C C A T GT C C C A G A A C T A C T G G T T T T T T G A C |
| | | G T C T G G G G C C A A G G C A C C A C G G T C A C C G T C T C C T C A |
| Clone 20 | 67 | C A G G T C C A A C T G G T G C A G T C T G G G G C T G A G G T G A A G |
| | | A A G C C T G G G G C T T C A G T G A A G A T G T C C T G C A A G G C T |
| | | T C T G G C T A C A G C T T C A C C A G C T A C T G G AT A A A C T G G |
| | | G T G A A G C A G A G G C C T G G A C A A G G C C T T G A G T G G A T T |
| | | G G A G A T G T G C A T C C T G G T A G A G G C G T G T C C A C A T A C |
| | | A A T G C T A A G T T C A A G A G C A GA G C C A C A C T G A C T C T G |
| | | G A C A C A T C C A T A A G C A C A G C C T A C A T G G A G C T C A G C |
| | | A G C C T G A G A T C T G A G G A C A C G G C G G T C T A T T A C T G T |
| | | G C C A G G T C C T C GG T G C A T G G C T A C T G G T T T T T T G A C |
| | | G T C T G G G G C C A A G G C A C C A C G G T C A C C G T C T C C T C A |
| Clone 21 | 68 | C A G G T C C A A C T G G T G C A G T C T G G G G C T G A G G T G A A G |
| | | A A G C C T G G G G C T T C A G T G A A G A T G T C C T G C A A G G C T |
| | | T C T G G C T A C A G C T T C A C C A G C T A C T G G AT A A A C T G G |
| | | G T G A A G C A G A G G C C T G G A C A A G G C C T T G A G T G G A T T |
| | | G G A G A T G T G C A T C C T G G T A G A G G C G T G T C C A C A T A C |
| | | A A T G C T A A G T T C A A G A G C A GA G C C A C A C T G A C T C T G |
| | | G A C A C A T C C A C A A G C A C A G C C T A C A T G G A G C T C A G C |
| | | A G C C T G A G A T C T G A G G A C A C G G C G G T C T A T T A C T G T |
| | | A G C A G A T C T C A TG G T A A C C G C C C G T G G T T T T A C G A C |
| | | T T C T G G G G C C A A G G C A C C A C G G T C A C C G T C T C C T C A |
| Clone 22 | 69 | C A G G T C C A A C T G G T G C A G T C T G G G G C T G A G G T G A A G |
| | | A A G C C T G G G G C T T C A G T G A A G A T G T C C T G C A A G G C T |
| | | T C T G G C T A C A G C T T C A C C A G C T A C T G G AT A A A C T G G |
| | | G T G A A G C A G A G G C C T G G A C A A G G C C T T G A G T G G A T T |
| | | G G A G A T G T G C A T C C T G G T A G A G G C G T G T C C A C A T A C |
| | | A A T G C T A A G T T C A A G A G C A GA G C C A C A C T G A C T C T G |
| | | G A C A C A T C C A T A A G C A C A G C C T A C A T G G A G C T C A G C |
| | | A G C C T G A G A T C T G A G G A C A C G G C G G T C T A T T A C T G T |
| | | G C C A G G T C C T C CC A C A A G G G C T A C T G G T T T T T T G A C |
| | | G T C T G G G G C C A A G G C A C C A C G G T C A C C G T C T C C T C A |
| Clone 24 | 70 | C A G G T C C A A C T G G T G C A G T C T G G G G C T G A G G T G A A G |
| | | A A G C C T G G G G C T T C A G T G A A G A T G T C C T G C A A G G C T |
| | | T C T G G C T A C A G C T T C A C C A G C T A C T G G AT A A A C T G G |
| | | G T G A A G C A G A G G C C T G G A C A A G G C C T T G A G T G G A T T |
| | | G G A G A T G T G C A T C C T G G T A G A G G C G T G T C C A C A T A C |
| | | A A T G C T A A G T T C A A G A G C A GA G C C A C A C T G A C T C T G |
| | | G A C A C A T C C A T A A G C A C A G C C T A C A T G G A G C T C A G C |
| | | A G C C T G A G A T C T G A G G A C A C G G C G G T C T A T T A C T G T |
| | | G C C A G G G T C T C CC C G A T G A G C T A C T G G T T T T T T G A C |
| | | G T C T G G G G C C A A G G C A C C A C G G T C A C C G T C T C C T C A |

TABLE 6
Humanized antibody IgG4 sequence

| | | Polypeptide |
|---|---|---|
| Constant regions | cDNA Sequence | sequence |
| IgG4 heavy chain | GCTTCCACCAAGGGCCCATCCGTCTTCCCCCTGGCGCCCTGCTCC | ASTKGPSVFPLAPCSR |
| SEQ ID NO: 73, 74 | AGGAGCACCTCCGAGAGCACAGCCGCCCTGGGCTGCCTGGTCAAG | STSESTAALGCLVKDY |
| | GACTACTTCCCCGAACCGGTGACGGTGTCGTGGAACTCAGGCGCC | FPEPVTVSWNSGALTS |
| | CTGACCAGCGGCGTGCACACCTTCCCGGCTGTCCTACAGTCCTCA | GVHTFPAVLQSSGLYS |
| | GGACTCTACTCCCTCAGCAGCGTGGTGACCGTGCCCTCCAGCAGC | LSSVVTVPSSSLGTKT |
| | TTGGGCACGAAGACCTACACCTGCAATGTAGATCACAAGCCCAGC | YTCNVDHKPSNTKVDK |
| | AACACCAAGGTGGACAAGAGAGTTGAGTCCAAATATGGTCCCCCA | RVESKYGPPCPPCPAP |
| | TGCCCACCATGCCCAGCACCTGAGTTCCTGGGGGGACCATCAGTC | EFLGGPSVFLFPPKPK |
| | TTCCTGTTCCCCCCAAAACCCAAGGACACTCTCATGATCTCCCGG | DTLMISRTPEVTCVVV |
| | ACCCCTGAGGTCACGTGCGTGGTGGTGGACGTGAGCCAGGAAGAC | DVSQEDPEVQFNWYVD |
| | CCCGAGGTCCAGTTCAACTGGTACGTGGATGGCGTGGAGGTGCAT | GVEVHNAKTKPREEQF |
| | AATGCCAAGACAAAGCCGCGGGAGGAGCAGTTCAACAGCACGTAC | NSTYRVVSVLTVLHQD |
| | CGTGTGGTCAGCGTCCTCACCGTCCTGCACCAGGACTGGCTGAAC | WLNGKEYKCKVSNKGL |
| | GGCAAGGAGTACAAGTGCAAGGTCTCCAACAAAGGCCTCCCGTCC | PSSIEKTISKAKGQPR |
| | TCCATCGAGAAAACCATCTCCAAAGCCAAAGGGCAGCCCCGAGAG | EPQVYTLPPSQEEMTK |
| | CCACAGGTGTACACCCTGCCCCCATCCCAGGAGGAGATGACCAAG | NQVSLTCLVKGFYPSD |
| | AACCAGGTCAGCCTGACCTGCCTGGTCAAAGGCTTCTACCCCAGC | IAVEWESNGQPENNYK |
| | GACATCGCCGTGGAGTGGGAGAGCAATGGGCAGCCGGAGAACAAC | TTPPVLDSDGSFFLYS |
| | TACAAGACCACGCCTCCCGTGCTGGACTCCGACGGCTCCTTCTTC | RLTVDKSRWQEGNVFS |
| | CTCTACAGCAGGCTAACCGTGGACAAGAGCAGGTGGCAGGAGGGG | CSVMHEALHNHYTQKS |
| | AATGTCTTCTCATGCTCCGTGATGCATGAGGCTCTGCACAACCAC | LSLSLGK |
| | TACACACAGAAGAGCCTCTCCCTGTCTCTGGGTAAATGA | |
| lgG4 Kappa | CGAACTGTGGCTGCACCATCTGTCTTCATCTTCCCGCCATCTGAT | RTVAAPSVFIFPPSDE |
| light chain | GAGCAGTTGAAATCTGGAACTGCCTCTGTTGTGTGCCTGCTGAAT | QLKSGTASVVCLLNNF |
| SEQ ID NO: 75, 76 | AACTTCTATCCCAGAGAGGCCAAAGTACAGTGGAAGGTGGATAAC | YPREAKVQWKVDNALQ |
| | GCCCTCCAATCGGGTAACTCCCAGGAGAGTGTCACAGAGCAGGAC | SGNSQESVTEQDSKDS |
| | AGCAAGGACAGCACCTACAGCCTCAGCAGCACCCTGACGCTGAGC | TYSLSSTLTLSKADYE |
| | AAAGCAGACTACGAGAAACACAAAGTCTACGCCTGCGAAGTCACC | KHKVYACEVTHQGLSS |
| | CATCAGGGCCTGAGCTCGCCCGTCACAAAGAGCTTCAACAGGGGA | PVTKSFNRGEC |
| | GAGTGTTAG | |

### Example 2

### Characterization of Selected Clones

### Methods and Materials

### Antibody Purification

Endotoxin-free DNA was prepared and Clones 4, 5, 14 and parental were transiently transfected into CHO cells using a MaxCyte STX® electroporation system (MaxCyte Inc., Gaithersburg, USA) with OC-400 processing assemblies. Following cell recovery, cells were diluted at 3×10⁶ cells/mL into CD Opti-CHO medium (ThermoFisher, Loughborough, UK) containing 8 mM L-Glutamine (ThermoFisher, Loughborough, UK) and 1×Hypoxanthine-Thymidine (ThermoFisher, Loughborough, UK). 24 hours post-transfection, the culture temperature was reduced to 32° C. and 1 mM sodium butyrate (Sigma, Dorset, UK) was added. Cultures were fed with 30% of culture volume of CHO CD Efficient Feed supplement B Invitrogen and 3.3% of culture volume of FunctionMAX TiterEnhancer. 7 days post transfections the feeding was as follow: 15% of culture volume of CHO CD Efficient Feed supplement B and 1.65% of culture volume of FunctionMAX TiterEnhancer. Transfections were cultured for up to 14 days prior to harvesting supernatants. Expression levels were tested and the titres were approximately 30 mg/L.

Antibodies from clones 4, 5, 14 and parental were purified from cell culture supernatants on Protein A sepharose columns (GE Healthcare, Little Chalfont, UK), buffer exchanged into PBS pH 7.2 and quantified by OD280 nm using an extinction coefficient based on the predicted amino acid sequence. Size exclusion chromatography using a Superdex™ 200 was performed on all the antibodies following protein A purification. After SEC all the antibodies showed less than 1% of aggregation.

### SPR Analysis of Antibody Binding to Cyclic Peptides, A-Beta Monomers and Oligomers

### A-beta Monomer and Oligomer Preparation:

Recombinant A-beta40 and 42 peptides (California Peptide, Salt Lake City Utah, USA) were dissolved in ice-cold hexafluoroisopropanol (HFIP). The HFIP was removed by evaporation overnight and dried in a SpeedVac centrifuge. To prepare monomers, the peptide film was reconstituted in DMSO to 5 mM, diluted further to 100 μM in dH2O and used immediately. Oligomers were prepared by diluting the 5 mM DMSO peptide solution in phenol red-free F12 medium (Life Technologies Inc., Burlington ON, Canada) to a final concentration of 100 μM and incubated for 24 hours to 7 days at 4° C. or were purchased from SynAging (Vandceuvre-lès-Nancy, France).

### SPR Analysis of Cyclic Peptide, A-Beta Monomer and Oligomer Binding:

All surface plasmon resonance (SPR) measurements were performed using a Molecular Affinity Screening System (MASS-1) (Sierra Sensors GmbH, Hamburg, Germany), an analytical biosensor that employs high intensity laser light and high speed optical scanning to monitor binding interactions in real time. In SPR direct binding assays, antibodies are covalently immobilized on individual flow cells of a High Amine Capacity (HAC) sensorchip (Sierra Sensors GmbH, Hamburg, Germany) and ligands flowed over the surface (BSA-conjugated peptides, A-beta42 Monomer and A-beta42 Oligomer). Each sample was diluted and injected in duplicate over the immobilized peptide and BSA reference surfaces, followed by injection of running buffer only for the dissociation phase. After every analytical cycle, the sensor chip surfaces were regenerated. Sensorgrams were double-referenced by subtracting out binding from the BSA reference surfaces and blank running buffer injections, and binding response report points collected in the dissociation phase.

### Brain Extract:

Brain tissues from 8 different human AD patients were obtained from Dr. Jiri Safar at Case Western Reserve University (Cleveland, Ohio, USA). The clinical diagnosis of AD was based on NINCDS-ADRDA criteria. Samples from frontal cortex were weighed and subsequently submersed in a volume of fresh, ice cold TBS buffer and EDTA-free protease inhibitor cocktail from Roche Diagnostics (Laval QC, Canada) such that the final concentration of brain tissue was 20% (w/v). Tissue was homogenized in this buffer using a mechanical probe homogenizer (3×30 sec pulses with 30 sec pauses in between, all performed on ice). TBS-homogenized samples were then subjected to ultracentrifugation for 90 min. Supernatants (soluble extracts) were collected, aliquoted and stored at −80° C. The protein concentration was determined using a bicinchoninic acid (BCA) protein assay. Pools of brain extracts from 8 patients were used in each analysis.

### Size Exclusion Chromatography

Pooled soluble brain extracts were injected at 0.5 ml/min through a Superdex™ 75 (10/300) HPLC column for 50 minutes and 0.25 ml fractions were collected. Molecular weight (MW) markers were run separately. Protein peaks were monitored by absorbance at O.D. 280 nm. Fractions corresponding to a MW of ˜8 kDa to ˜70 kDa were pooled into a low molecular weight (LMW) fraction. Aβ monomers (MW ˜4.5 kDa) were excluded from the LMW fraction. Fractions corresponding to a MW of >70 kDa to ˜700 kDa were pooled into a high molecular weight (HMW) fraction. The LMW and HMW fractions were concentrated and total protein concentration was determined in a BCA assay. The fractions were then diluted in PBS-EP, BSA (2 mg/ml) buffer to 100 μg/ml for surface plasmon resonance (SPR) analysis.

### Surface Plasmon Resonance Analysis

Surface plasmon resonance measurements were performed as described above.

Purified antibodies were immobilized on sensorchips. HMW and LMW fractions from pooled soluble human AD brain extracts (100 μg/ml) were injected over the surfaces followed by a dissociation phase. Sensorgrams were double-reference subtracted.

### Immunohistochemistry

Fresh frozen AD brain sections were exposed to antigen retrieval citrate buffer (Target Retrieval Solution, Dako, Santa Clara Calif., USA) for 20 min and incubated in a humidified chamber with serum-free protein blocking reagent (Dako) for 1 h to block non-specific staining. The sections were incubated overnight at 4° C. with primary antibodies at 1 μg/ml and washed 3 times for 5 min in Tris-buffered saline containing 0.1% TritonX-100 (TBS-T) buffer. Secondary HRP-conjugated rabbit anti-human IgG (0.4 μg/ml; Abcam, San Francisco Calif., USA) antibody was added to the sections and incubated for 1 hour, followed by 3 washes in TBS-T buffer. Secondary antibody was also added to sections that were exposed to human IgG4 isotype control as the primary antibody. The HRP enzyme substrate, biaminobezidine (DAB) chromogen reagent (Vector Laboratories, Burlingame Calif., USA), was then added to the sections followed by rinsing with distilled water. The sections were counterstained with haematoxylin QS (Vector Laboratories, Burlingame Calif., USA). The slides were examined under a light microscope (Zeiss Axiovert 200M, Carl Zeiss Toronto ON, Canada) and representative images were captured using a Leica DC300 digital camera and software (Leica Microsystems Canada Inc., Vaughan ON, Canada).

### Results

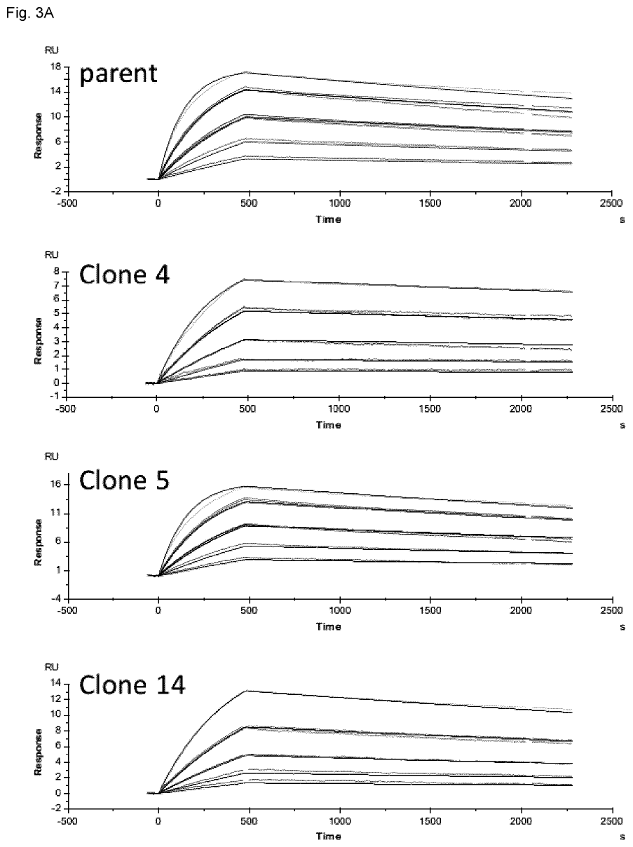

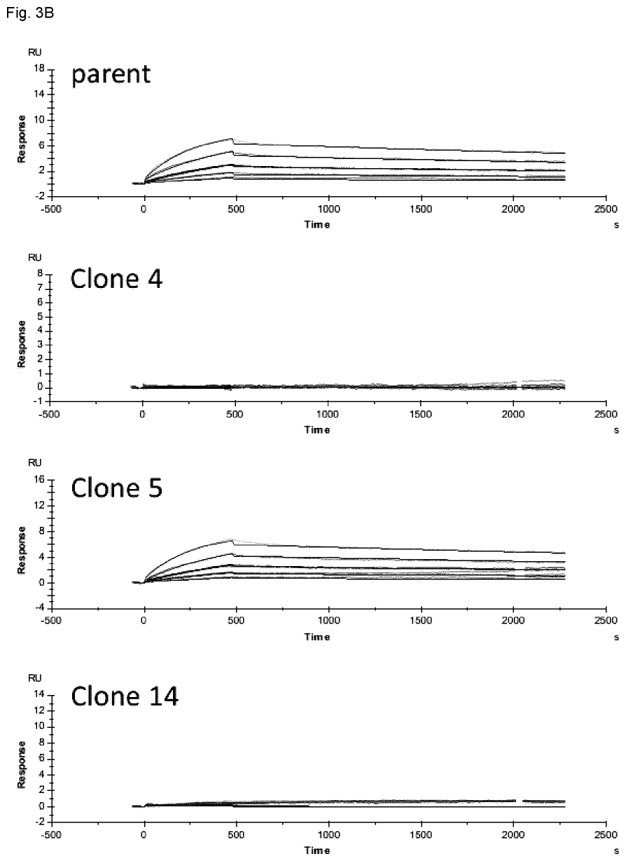

As shown in FIG. 3B and Table 7, no appreciable binding for linear peptide was observed for purified clones 4 and 14. As shown in Table 7, all tested antibodies showed selective binding for synthetic AβO vs Aβ monomers as assessed by SPR as well as lack of plaque binding by immunohistochemistry on frozen AD brain sections.

### SPR Analysis of Cyclic Peptide, A-Beta Monomer and Oligomer Binding:

As shown in Table 7 below, none of the antibody clones tested bound A-beta monomers. Antibody clones 4, 5, and 14, bound the stable A-beta 42 oligomers with binding response units (BRUs) comparable or greater than the parental antibody.

### Binding to Low Molecular Weight Toxic Aβ Oligomer-Enriched Soluble AD Brain Extracts

Examination of soluble Aβ species in AD brain extracts by several investigators has indicated that the neurotoxic activity resides primarily in the low molecular weight (LMW) fraction of AβO (dimers, trimers, tetramers, dodecamers) while high molecular weight (HMW) aggregates are largely inert though they reportedly can dissociate into LMW species. Therefore, size exclusion chromatography (SEC) of pooled soluble extracts from AD brains was performed. SEC fractionation of soluble AD brain extract gave rise to a highly reproducible pattern with protein peaks in the MW regions expected to contain LMW AβO. Fractions corresponding to ˜8-70 kDa were pooled into a LMW fraction expected to contain AβO in the dimer to dodecamer range and excluding monomers. Fractions corresponding to >70-700 kDa were pooled into a HMW fraction.

Binding of immobilized Aβ-directed antibodies to the LMW and HMW fractions of soluble AD brain extract was assessed by SPR. In 3 separate studies, Clones 4, 5 and 14 showed overall preferential binding to the toxic oligomer-enriched LMW fraction vs HMW fraction, comparable to the results obtained with parental huPMN310.

Aβ aggregates are present in the LMW/HMW fractions but to rule out the possibility that the antibodies may have been binding to other protein(s) also present in brain extract, a sandwich SPR assay was conducted whereby the material captured by immobilized antibodies was subsequently exposed to a detector antibody. Aducanumab was chosen as the detector antibody as it is known to be specific for Aβ and was expected to bind/detect material captured by immobilized aducanumab, thereby acting as a positive control. As shown in Table 7, both the HMW and LMW material captured by the test antibodies was detected by aducanumab (with the exception of LMW material captured by clone 5) thereby confirming binding of the antibodies to AβO in the brain fractions. Overall, clone 4 and parental huPMN310 showed the highest degree of direct capture and detection.

### Immunohistochemistry on Formalin Fixed Tissues:

As shown in Table 7 below, using AD brain tissue, the tested antibodies were negative for specific staining of senile plaque amyloid. Bapineuzumab, used as the positive control, showed strong plaque staining clearly differentiated from the negative IgG4 isotype control.

TABLE 7
Properties of selected purified antibody clones

| Properties | Parental | Clone 4 | Clone 5 | Clone 14 |
|---|---|---|---|---|
| Binding to cyclic peptide | + | + | + | + |
| KD (M) | 2.74 × 10⁻¹¹ | 2.35 × 10⁻¹¹ | 6.47 × 10⁻¹¹ | 2.89 × 10⁻¹¹ |
| Ka (1/Ms) | 5.65 × 10⁶ | 2.94 × 10⁶ | 2.04 × 10⁶ | 5.26 × 10⁶ |
| Kd (1/s) | 1.55 × 10⁻⁴ | 6.90 × 10⁻⁵ | 1.32 × 10⁻⁴ | 1.52 × 10⁻⁴ |
| Binding to linear peptide | +/− | Negligible | +/− | Negligible |
| KD (M) | 1.03 × 10⁻¹⁰ | — | 9.24 × 10⁻¹¹ | — |
| Ka (1/Ms) | 1.50 × 10⁶ | — | 1.49 × 10⁶ | — |
| Kd (1/s) | 1.55 × 10⁻⁴ | — | 1.38 × 10⁻⁴ | — |
| Binding to monomers (BRU) | Neg | Neg | Neg | Neg |
| Binding to oligomer prep (BRU) | 85.57 | 57.31 | 222.92 | 30.76 |
| Binding to commercial oligomer | 3 | 22.56 | 32.27 | 22.33 |
| (BRU) | | | | |
| Binding to LMW/HMW (BRU & ratio) | | | | |
| Study 1 | ~27/27 | ~28/21 | ~15/15 | ~18/16 |
| | 1.0 | 1.32 | 1.0 | 1.1 |
| Study 2 | 17.7/13.47 | 17.33/11.16 | 9.42/6.57 | 11.12/7.55 |
| | 1.31 | 1.55 | 1.43 | 1.47 |
| Study 3 | 7.6/5 | 10.11/6.77 | 9.45/7.88 | 6.34/4.38 |
| | 1.52 | 1.54 | 1.2 | 1.45 |
| Bound Ab detection by aducanumab | | | | |
| LMW (BRU) | ++ (5.8) | ++ (7.7) | − (0) | + (4.4) |
| HMW (BRU) | ++ (8.3) | ++ (10.4) | +/− (1.25) | + (5.7) |
| IHC plaque binding | Neg | Neg | Neg | Neg |

### Example 3

### Inhibition of Oligomer Propagation

The biological functionality of the parental antibodies was tested in vitro by examining their effects on Amyloid Beta (Aβ) aggregation using the Thioflavin T (ThT) binding assay. Aβ aggregation is induced by and propagated through nuclei of preformed small Aβ oligomers, and the complete process from monomeric Aβ to soluble oligomers to insoluble fibrils is accompanied by concomitantly increasing beta sheet formation. This can be monitored by ThT, a benzothiazole salt, whose excitation and emission maxima shifts from 385 to 450 nm and from 445 to 482 nm respectively when bound to beta sheet-rich structures and resulting in increased fluorescence. Briefly, Aβ 1-42 (Bachem Americas Inc., Torrance, Calif.) was solubilized, sonicated, diluted in Tris-EDTA buffer (pH7.4) and added to wells of a black 96-well microtitre plate (Greiner Bio-One, Monroe, N.C.) to which equal volumes of cyclopeptide raised antibody or irrelevant mouse IgG antibody isotype controls are added, resulting in a 1:5 molar ratio of Aβ1-42 peptide to antibody. ThT was added and plates incubated at room temperature for 24 hours, with ThT fluorescence measurements (excitation at 440 nm, emission at 486 nm) recorded every hour using a Wallac Victor3v 1420 Multilabel Counter (PerkinElmer, Waltham, Mass.). Fluorescent readings from background buffer were subtracted from all wells, and readings from antibody only wells are further subtracted from the corresponding wells.

Aβ42 aggregation, as monitored by ThT fluorescence, demonstrated a sigmoidal shape characterized by an initial lag phase with minimal fluorescence, an exponential phase with a rapid increase in fluorescence and finally a plateau phase during which the Aβ molecular species are at equilibrium and during which there is no increase in fluorescence. Co-incubation of Aβ42 with an irrelevant mouse antibody has been shown not have any significant effect on the aggregation process. In contrast, co-incubation of Aβ42 with the test antibodies inhibited the aggregation process. Inhibition seen with the parental antibodies is demonstrated in WO 2018/014126 and WO 2019/014768. The antibodies described herein are expected to have similar or better inhibition.

### Example 4

### Toxicity Inhibition Assay

The inhibition of toxicity of A-beta42 oligomers by parental antibodies was tested in a rat primary cortical neuron assay.

Antibody and control IgG were each adjusted to a concentration such as 2 mg/mL. Various molar ratios of A-beta oligomer and antibody were tested along with a vehicle control, A-beta oligomer alone and a positive control such as the neuroprotective peptide humanin HNG. Following preincubation for 10 minutes at room temperature, the volume was adjusted to 840 microlitres with culture medium. The solution was incubated for 5 min at 37 C. The solution was then added directly to the primary cortical neurons and cells are incubated for 24 h. Cell viability was determined using the MTT assay.

The effect of antibody on neuronal cell viability in the presence and absence of A-beta oligomers was compared.

As demonstrated in WO 2018/014126, in the absence of A-beta oligomers, the parental antibody alone had no effect on neuronal cell viability. When incubated in the presence of A-beta oligomers, the parental antibody inhibited A-beta oligomer-induced neuronal death at all molar ratios tested. The antibodies described herein are expected to have similar or better inhibition.

### Example 5

### In Vivo Toxicity Inhibition Assay

The inhibition of toxicity of A-beta42 oligomers by the antibodies can be tested in vivo in mouse behavioral assays.

### Novel Object Recognition (NOR)

The Novel Object Recognition (NOR) model utilizes the normal behavior of rodents to investigate novel objects for a significantly longer time than known objects. This test assesses recognition memory for items and its human equivalent is the visual pairwise-comparison (VPC). Recognition of objects is mediated by the perirhinal cortex in rodents, primates and humans. AD pathology develops first in the perirhinal and enthorinal cortex before the hippocampus. The VPC task detects memory deficit in mild cognitive impairment (MCI) and conversion from MCI to AD is predicted by this task (Zola S M et al, 2016).

As demonstrated in WO 2018/014126, co-injection of parental antibody with AβO reduces or prevents AβO-induced cognitive deficits in the NOR test. Mice co-administered the parental antibody and AβO exhibit a mean discrimination index not different from control mice but different from AβO-injected mice. The parental antibody offers protection against AβO-induced cognitive deficits. The antibodies described herein are expected to offer similar or better protection against AβO-induced cognitive deficits.

While the present application has been described with reference to what are presently considered to be the preferred examples, it is to be understood that the application is not limited to the disclosed examples. To the contrary, the application is intended to cover various modifications and equivalent arrangements included within the spirit and scope of the appended claims.

All publications, patents and patent applications are herein incorporated by reference in their entirety to the same extent as if each individual publication, patent or patent application was specifically and individually indicated to be incorporated by reference in its entirety. Specifically, the sequences associated with each accession numbers provided herein including for example accession numbers and/or biomarker sequences (e.g. protein and/or nucleic acid) provided in the Tables or elsewhere, are incorporated by reference in its entirely.

The scope of the claims should not be limited by the preferred embodiments and examples, but should be given the broadest interpretation consistent with the description as a whole.

### CITATIONS FOR REFERENCES REFERRED TO IN THE SPECIFICATION

Giulian D, Haverkamp L J, Yu J, Karshin W, Tom D, Li J, Kazanskaia A, Kirkpatrick J, Roher A E. The HHQK domain of β-amyloid provides a structural basis for the immunopathology of Alzheimer's disease, J. BiolChem. 1998, 273(45), 29719-26.
Winkler K, Scharnagl H, Tisljar U, Hoschützky H, Friedrich I, Hoffmann M M, Hüttinger M, Wieland H, März W. Competition of Aβ amyloid peptide and apolipoprotein E for receptor-mediated endocytosis. J. Lipid Res. 1999, 40(3), 447-55.
Crespi, G., Hermans, S., Parker, M. et al. Molecular basis for mid-region amyloid-β capture by leading Alzheimer's disease immunotherapies. Sci Rep 5, 9649 (2015).
Yu Y Z, Wang W B, Chao A, Chang Q, Liu S, Zhao M, et al. Strikingly reduced amyloid burden and improved behavioral performance in Alzheimer's disease mice immunized with recombinant chimeric vaccines by hexavalent foldable Ab 1-15 fused to toxin-derived carrier proteins. J Alzheimer's Dis 2014; 41:243-60.
Wang, H C; Yu, Y Z; Liu, S; Zhao, M and Q Xu, Peripherally administered sera antibodies recognizing amyloid-oligomers mitigate Alzheimer's disease-like pathology and cognitive decline in aged 3× Tg-AD mice, Vaccine 2016.
Zola S M, Manzanares C M, Clopton P, Lah J J, Levey A I. A behavioral task predicts conversion to mild cognitive impairment and Alzheimer's disease. Am J Alzheimer's Dis & other dementia. 2013, 28(2), 179-184.
Peters S J et al. Engineering an Improved IgG4 Molecule with Reduced Disulfide Bond Heterogeneity and Increased Fab Domain Thermal Stability, The Journal of Biological Chemistry (2012) 287, 24525-24533.

1. An antibody comprising a light chain variable region and a heavy chain variable region, the heavy chain variable region comprising complementarity determining regions CDR-H1, CDR-H2, and CDR-H3, the CDR-H3 having a sequence selected from any one of SEQ ID NOs: 31-36, 38-40 or 42-50.

2. The antibody of claim 1, wherein the light chain variable region comprises complementarity determining regions CDR-L1, CDR-L2, and CDR-L3, the CDR-L1 comprising the sequence of SEQ ID NO: 8, the CDR-L2 comprising the sequence of SEQ ID NO: 9, and the CDR-L3 comprising the sequence of SEQ ID NO: 10, optionally wherein the light chain variable region comprises i) an amino acid having the sequence of SEQ ID NO: 4, ii) an amino acid sequence with at least 80%, at least 90%, or at least 95% sequence identity to SEQ ID NO: 4, wherein the CDR-L1, CDR-L2 and CDR-L3 sequences are as set forth in SEQ ID NOs: 8, 9 and 10, respectively, or iii) a conservatively substituted amino acid sequence of i) wherein the CDR-L1, CDR-L2 and CDR-L3 sequences are as set forth in SEQ ID NOs: 8, 9 and 10, respectively.

3. (canceled)

4. The antibody of claim 1, wherein the heavy chain variable region comprises complementary determining regions CDR-H1 and CDR-H2, the CDR-H1 comprising the sequence of SEQ ID NO:5, and the CDR-H2 comprising the sequence of SEQ ID NO: 6, optionally wherein the heavy chain variable region comprises i) an amino acid sequence as set forth in any one of SEQ ID NOs: 11-16, 18-20 or 22-30, ii) an amino acid sequence with at least 80%, at least 90%, or at least 95% sequence identity to any one of SEQ ID NOs: 11-16, 18-20 or 22-30, wherein the CDR-H3 has a sequence as set forth in SEQ ID NOs: 31-36, 38-40 or 42-50, respectively, or iii) a conservatively substituted amino acid sequence of i), wherein the CDR-H3 has a sequence as set forth in SEQ ID NOs: 31-36, 38-40 or 42-50, respectively.

5. The antibody of claim 1, wherein the heavy chain variable region amino acid sequence is encoded by a nucleotide sequence as set forth in any one of SEQ ID Nos: 51-56, 58-60 or 62-70; or a codon degenerate or optimized version thereof, optionally wherein the heavy chain variable region amino acid sequence is encoded by a nucleotide sequence as set forth in any one of SEQ ID Nos: 51, 53, 54, 58-60, 62, 66-68 or 70; or a codon degenerate or optimized version thereof, optionally wherein the heavy chain variable region amino acid sequence is encoded by a nucleotide sequence as set forth in SEQ ID Nos: 53 or 62; or a codon degenerate or optimized version thereof.

6. The antibody of claim 1, wherein the CDR-H3 has a sequence selected from any one of SEQ ID NOs: 31, 33, 34, 38-40, 42, 46-48 or 50, optionally wherein the heavy chain variable region comprises i) an amino acid sequence as set forth in any one of SEQ ID NOs: 11, 13, 14, 18-20, 26-28 or 30, ii) an amino acid sequence with at least 80%, at least 90%, or at least 95% sequence identity to any one of SEQ ID NOs: 11, 13, 14, 18-20, 26-28 or 30, wherein the CDR-H3 has a sequence as set forth in SEQ ID NOs: 31, 33, 34, 38-40, 42, 46-48 or 50, respectively, or iii) a conservatively substituted amino acid sequence of i) wherein the CDR-H3 has a sequence as set forth in SEQ ID NOs: 31, 33, 34, 38-40, 42, 46-48 or 50, respectively.

7. (canceled)

8. (canceled)

9. The antibody of claim 1, wherein the CDR-H3 has a sequence of SEQ ID NOs: 33 or 42, optionally wherein the heavy chain variable region comprises i) an amino acid sequence as set forth in SEQ ID NOs: 13 or 22, ii) an amino acid sequence with at least 80%, at least 90%, or at least 95% sequence identity to SEQ ID NOs: 13 or 22, wherein the CDR-H3 has a sequence as set forth in SEQ ID NOs: 33 or 42, respectively, or iii) a conservatively substituted amino acid sequence of i) wherein the CDR-H3 has a sequence as set forth in SEQ ID NOs: 33 or 42, respectively.

10. (canceled)

11. (canceled)

12. An affinity matured antibody that competes for binding to cyclo(CGHHQKG) (SEQ ID NO: 2) peptide and/or oligomeric A-beta with a reference antibody, the reference antibody comprising CDR-H1, CDR-H2, CDRH3, CDR-L1, CDR-L2 and CDR-L3 regions as set forth in SEQ ID NOs: 5 to 10, respectively, or comprising a light chain variable region and a heavy chain variable region as set forth in SEQ ID NOs: 3 and 4, respectively, preferably wherein the antibody has at least 80%, at least 90%, or at least 95% sequence identity to the reference antibody, with the proviso that the affinity matured antibody is not the reference antibody, optionally wherein the antibody has greater differential binding activity for cyclo(CGHHQKG) (SEQ ID NO: 2) peptide over linear(CGHHQKG) (SEQ ID NO: 2) peptide than the reference antibody by at least or about 3-fold, at least or about 4-fold, at least or about 5-fold, at least or about 6-fold, at least or about 7-fold, at least or about 10-fold, at least or about 15-fold, at least or about 20-fold or at least or about 25-fold.

13. (canceled)

14. The antibody of claim 1, wherein the antibody has a KD of at least or about 2.5×10⁻¹¹ for a cyclo(CGHHQKG) (SEQ ID NO: 2) peptide.

15. (canceled)

16. The antibody of claim 1, wherein the antibody preferentially binds cyclo(CGHHQKG) (SEQ ID NO: 2) peptide over linear(CGHHQKG) (SEQ ID NO: 2) peptide by at least or about 5-fold, at least or about 7-fold, at least or about 8-fold, at least or about 9-fold, at least or about 10-fold, at least or about 100-fold, at least or about 200-fold, at least or about 500-fold or at least or about 1000-fold.

17. The antibody of claim 1, wherein the antibody is an antibody binding fragment selected from Fab, Fab′, F(ab′)2, scFv, dsFv, ds-scFv, dimers, nanobodies, minibodies, diabodies, and multimers thereof, or wherein the antibody is a single chain antibody.

18. The antibody of claim 17, wherein the antibody binding fragment is a Fab fragment, optionally comprising the heavy chain variable region of any one of SEQ ID NOs: 11-16, 18-20 or 22-30, preferentially any one of SEQ ID NOs: 11, 13, 14, 18-20, 26-28 or 30, or more preferentially SEQ ID NOs: 13 or 22.

19. The antibody of claim 1, wherein the antibody is IgG1 or IgG4.

20. (canceled)

21. (canceled)

22. The antibody of claim 17, wherein the antibody comprises SEQ ID NOs: 74 and/or 76, and/or the CH1 and heavy chain constant domain 2 (CH2) of SEQ ID NO: 74 or a conservatively substituted amino acid sequence of any of the foregoing or a sequence with at least 80%, 90% or 95% sequence identity to any of the foregoing.

23. The antibody of claim 1 for inhibiting A-beta oligomer propagation in a subject or treating Alzheimer's disease (AD) and/or other A-beta amyloid related diseases.

24. (canceled)

25. (canceled)

26. An immunoconjugate comprising the antibody of claim 1 and a detectable label or cytotoxic agent, optionally wherein the detectable label comprises a positron emitting radionuclide, optionally for use in subject imaging such as PET imaging.

27. (canceled)

28. A composition comprising the antibody of claim 1, or the immunoconjugate comprising said antibody, optionally with a diluent.

29. A vector or nucleic acid molecule encoding the antibody of claim 1.

30. (canceled)

31. A cell expressing the antibody of claim 1.

32. A kit comprising the antibody of claim 1, the immunoconjugate comprising said antibody, the nucleic acid molecule encoding said antibody, the vector expressing said antibody.

33. A method for determining if the biological sample contains A-beta oligomer, the method comprising:
a. contacting the sample with the antibody of claim 1 or an immunoconjugate comprising said antibody that is specific and/or selective for A-beta oligomers under conditions permissive for forming an antibody: A-beta oligomer complex; and
b. detecting the presence of any complex;
wherein the presence of detectable complex is indicative that the sample may contain A-beta oligomer.

34. (canceled)

35. (canceled)

36. (canceled)

37. A method of measuring a level of oligomeric A-beta in a subject, the method comprising administering to a subject at risk or suspected of having or having AD, an immunoconjugate comprising an antibody of claim 1, wherein the antibody is conjugated to a detectable label, optionally wherein the label is a positron emitting radionuclide; and detecting the label, optionally quantitatively detecting the label.

38. (canceled)

39. (canceled)

40. A method of treating AD and/or other A-beta amyloid related diseases, the method comprising administering to a subject in need thereof i) an effective amount of an antibody or immunoconjugate of claim 1, or a pharmaceutical composition comprising said antibody or said immunoconjugate; or 2) a nucleic acid or vector comprising a nucleic acid encoding said antibody, to a subject in need thereof.

41. The method of claim 40, wherein a biological sample from the subject to be treated is assessed for the presence or levels of A-beta.

42. The method of claim 40, wherein the antibody, immunoconjugate, composition or nucleic acid or vector is administered directly to the brain or other portion of the central nervous system (CNS), or systemically.

43. (canceled)
